(19) United States
(12) Patent Application Publication
(10) Pub. No.: US 2025/0242019 A2
(43) Pub. Date: Jul. 31, 2025

(54) METHODS FOR TREATING MULTIPLE MYELOMA

(71) Applicant: Janssen Biotech, Inc., Horsham, PA (US)

(72) Inventors: Rachel KOBOS, Bridgewater, NJ (US); Arnob BANERJEE, Baltimore, MD (US); Jenna GOLDBERG, Livingston, NJ (US); Katherine CHASTAIN, Kansas City, MO (US)

(21) Appl. No.: 18/639,242

(22) Filed: Apr. 18, 2024

Related U.S. Application Data
(60) Provisional application No. 63/568,836, filed on Mar. 22, 2024.
(60) Provisional application No. 63/594,786, filed on Oct. 31, 2023.
(60) Provisional application No. 63/505,587, filed on Jun. 1, 2023.
(60) Provisional application No. 63/504,164, filed on May 24, 2023.
(60) Provisional application No. 63/497,122, filed on Apr. 19, 2023.
(60) Provisional application No. 63/497,121, filed on Apr. 19, 2023.

(51) Int. Cl. A61K 39/395; A61K 39/00; C07K 16/28

(52) U.S. Cl. CPC A61K 39/39558; A61K 39/39541; C07K 16/2809; C07K 16/2878; C07K 16/2896; A61K 2039/507; C07K 2317/24; C07K 2317/31

(57) ABSTRACT

Embodiments of the present invention relate to methods of treating multiple myeloma in a subject in need thereof comprising administering to the subject a BCMAxCD3 bispecific antibody on a bi-weekly dosing schedule.

### CROSS REFERENCE TO RELATED APPLICATIONS

This application claims the benefit of U.S. Provisional Application Ser. No. 63/497,121 filed on Apr. 19, 2023; 63/497,122 filed on Apr. 19, 2023; 63/504,164 filed on May 24, 2023; 63/505,587 filed on Jun. 1, 2023; 63/594,786 filed on Oct. 31, 2023; and 63/568,836 filed on Mar. 22, 2024. The entire contents of the aforementioned applications are incorporated herein by reference in their entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “258199061501 (JBI6802USNP1)_Sequence_Listing.xml”, was created on Apr. 18, 2024 and is 26,376 bytes in size.

### FIELD OF THE INVENTION

Methods of treating multiple myeloma are disclosed.

### BACKGROUND OF THE INVENTION

Multiple myeloma (MM) is a cancer of the plasma cells. Mechanistically, multiple myeloma is characterized by production of monoclonal proteins (M-proteins) comprised of pathological immunoglobulins or fragments of such, which have lost their function. The proliferation of multiple myeloma cells leads to subsequent displacement from the normal bone marrow niche, while overproduction of M-proteins causes characteristic osteolytic lesions, increased susceptibility to infections, hypercalcemia, renal insufficiency or failure, and neurological complications.

Treatment options for multiple myeloma have improved over time and vary depending on the aggressiveness of the disease, underlying prognostic factors, physical condition of the patient, and existing comorbidities. Therapeutic options include proteasome inhibitors (PIs), immunomodulatory drugs (IMiDs), alkylating agents, monoclonal antibodies (mAbs), antibody drug conjugate, histone deacetylase inhibitor, nuclear protein export inhibitor, chimeric antigen receptor (CAR) T cell therapy and stem cell transplantation.

Despite these therapeutic achievements, the disease recurs and is associated with additional risk factors (e.g., comorbidities or increasing age), thus warranting the need for novel therapeutic approaches, such as new dosage and treatment regimens. In particular in the elderly population, for which stem cell transplantation is often not a viable option, and in patients with refractory disease who exhausted several therapies, multiple myeloma remains an incurable malignancy and an unmet medical need with significant morbidity and mortality. In particular, there remains a need for therapeutic regimens that achieve rapid, deep and durable clinical responses, while providing manageable safety profiles.

### SUMMARY OF THE INVENTION

An embodiment of the present invention provides a method of treating multiple myeloma in a subject in need thereof, the method comprising administering a therapeutically effective amount of a BCMAxCD3 bispecific antibody on a bi-weekly (Q2W) dosing schedule.

Another embodiment of the present invention provides a method of treating multiple myeloma in a subject in need thereof, the method comprising administering a therapeutically effective amount of a BCMAxCD3 bispecific antibody on a Q4W dosing schedule.

In certain embodiments, a method of treating multiple myeloma in a subject in need thereof comprises administering to the subject a therapeutically effective amount of a BCMAxCD3 bispecific antibody (e.g., teclistamab) on a bi-weekly dosing schedule (Q2W) and/or a monthly dosing schedule (Q4W), wherein the subject has been diagnosed with multiple myeloma.

In certain embodiments, a method of treating multiple myeloma in a subject in need thereof comprises administering to the subject treatment doses of a BCMAxCD3 bispecific antibody on a bi-weekly dosing schedule (Q2W).

In certain embodiments, the BCMAxCD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

In certain embodiments, the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.

In certain embodiments, the BCMAxCD3 bispecific antibody is an IgG1, an IgG2, an IgG3 or an IgG4 isotype.

In certain embodiments, the BCMAxCD3 bispecific antibody is an IgG4 isotype.

In certain embodiments, the BCMAxCD3 bispecific antibody comprises one or more substitutions in its Fc region.

In certain embodiments, the BCMAxCD3 bispecific antibody is an IgG4 isotype and comprises S228P, F234A and L235A substitutions in its Fc region (according to EU numbering).

In certain embodiments, the BCMAxCD3 bispecific antibody is an IgG4 isotype and comprises S228P, F234A, L235A F405L and R409K substitutions in its Fc region (according to EU numbering).

In certain embodiments, the Fc region of the BCMA-binding arm comprises S228P, F234A and L235A substitutions in its Fc region (according to EU numbering).

In certain embodiments, the Fc region of the CD3-binding arm comprises S228P, F234A, L235A, F405L, and R409K substitutions in its Fc region (according to EU numbering).

In certain embodiments, the BCMAxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.

In certain embodiments, the BCMAxCD3 bispecific antibody comprises a first heavy chain (HC1) having at least 90% identity to the amino acid sequence of SEQ ID NO: 12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.

In certain embodiments, the BCMAxCD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.

In certain embodiments, the BCMAxCD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.

In certain embodiments, the BCMAxCD3 bispecific antibody is teclistamab.

In certain embodiments, the subject has relapsed or refractory multiple myeloma myeloma (e.g., the subject has had been treated with from 2 to 14 prior lines of therapy).

In certain embodiments, the subject has received at least three prior lines of therapy.

In certain embodiments, the subject has received at least four prior lines of therapy.

In certain embodiments, the subject has received at least five prior lines of therapy (penta-drug exposed).

In certain embodiments, the subject has received at least three prior lines of therapy, including a proteasome inhibitor, an immunomodulatory agent and an anti-CD38 monoclonal antibody.

In certain embodiments, the subject has received at least four prior lines of therapy, including a proteasome inhibitor, an immunomodulatory agent and an anti-CD38 monoclonal antibody.

In certain embodiments, the method comprises subcutaneously administering treatment doses of the BCMAxCD3 bispecific antibody on the bi-weekly dosing schedule (Q2W).

In certain embodiments, the method comprises subcutaneously administering to the subject one or more step-up doses of the BCMAxCD3 bispecific antibody prior to administering the first treatment dose of the BCMAxCD3 bispecific antibody.

In certain embodiments, the method comprises subcutaneously administering the BCMAxCD3 bispecific antibody on the bi-weekly dosing schedule (Q2W) at a treatment dose of from about 1500 μg/kg to about 3000 μg/kg.

In certain embodiments, the method comprises subcutaneously administering the BCMAxCD3 bispecific antibody on the bi-weekly dosing schedule (Q2W) at a treatment dose of about 1500 μg/kg.

In certain embodiments, the method comprises subcutaneously administering the BCMAxCD3 bispecific antibody on the bi-weekly dosing schedule (Q2W) at a treatment dose of about 3000 μg/kg.

In certain embodiments, the method comprises subcutaneously administering to the subject at least one treatment dose of the BCMAxCD3 bispecific antibody on a weekly dosing schedule (QW) and subsequently administering to the subject treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least one 28-day cycle of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least two 28-day cycles of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least three 28-day cycles of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least four 28-day cycles of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least five 28-day cycles of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least six 28-day cycles of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least six months, and subsequently administering treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a partial response, a very good partial response, a complete response or a stringent complete response, as determined by IMWG response criteria, and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a very good partial response, a complete response or a stringent complete response, as determined by IMWG response criteria, and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a complete response or a stringent complete response, as determined by IMWG response criteria, and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a partial response, a very good partial response, a complete response or a stringent complete response after four or more cycles of treatment, as determined by IMWG response criteria, and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a partial response, a very good partial response, a complete response or a stringent complete response for six or more months, as determined by IMWG response criteria, and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a complete response or a stringent complete response for six or more months, as determined by IMWG response criteria, and subsequently administering to the subject treatment doses of the BCMAxCD3 bispecific antibody on the bi-weekly dosing schedule (Q2W).

In certain embodiments, each treatment dose amount of the BCMAxCD3 bispecific antibody administered on the weekly dosing schedule (QW) is the same treatment dose amount of the BCMAxCD3 bispecific antibody administered on the bi-weekly dosing schedule (Q2W).

In certain embodiments, the method comprises subcutaneously administering the BCMAxCD3 bispecific antibody on the weekly dosing schedule (QW) at a treatment dose of from about 1500 μg/kg to about 3000 μg/kg.

In certain embodiments, the method comprises subcutaneously administering the BCMAxCD3 bispecific antibody on the weekly dosing schedule (QW) at a treatment dose of about 1500 μg/kg.

In certain embodiments, the method comprises subcutaneously administering the BCMAxCD3 bispecific antibody on the weekly dosing schedule (QW) at a treatment dose of about 3000 μg/kg.

In certain embodiments, the method comprises subcutaneously administering the BCMAxCD3 bispecific antibody on the weekly dosing schedule (QW) at a treatment dose of about 1500 μg/kg, and then subcutaneously administering the BCMAxCD3 bispecific antibody on the bi-weekly dosing schedule (Q2W) at a treatment dose of about 1500 μg/kg.

In certain embodiments, the method comprises subcutaneously administering the BCMAxCD3 bispecific antibody on the weekly dosing schedule (QW) at a treatment dose of about 1500 μg/kg, and then subcutaneously administering the BCMAxCD3 bispecific antibody on the bi-weekly dosing schedule (Q2W) at a treatment dose of about 3000 μg/kg.

In certain embodiments, the method comprises subcutaneously administering the BCMAxCD3 bispecific antibody on the bi-weekly dosing schedule (Q2W), and then subcutaneously administering the BCMAxCD3 bispecific antibody on a monthly dosing schedule (Q4W).

In certain embodiments, the method comprises subcutaneously administering the BCMAxCD3 bispecific antibody on a weekly dosing schedule (QW), and then subcutaneously administering the BCMAxCD3 bispecific antibody on the bi-weekly dosing schedule (Q2W), and then subcutaneously administering the BCMAxCD3 bispecific antibody on a monthly dosing schedule (Q4W).

In certain embodiments, the method comprises subcutaneously administering the BCMAxCD3 bispecific antibody on the bi-weekly dosing schedule (Q2W), and if the subject achieves a partial response, a very good partial response, a complete response or a stringent complete response, then subcutaneously administering the BCMAxCD3 bispecific antibody on a monthly dosing schedule (Q4W).

In certain embodiments, the method comprises subcutaneously administering the BCMAxCD3 bispecific antibody on the bi-weekly dosing schedule (Q2W), and if the subject achieves a complete response or a stringent complete response, then subcutaneously administering the BCMAxCD3 bispecific antibody on a monthly dosing schedule (Q4W).

In certain embodiments, the method comprises subcutaneously administering the BCMAxCD3 bispecific antibody on the bi-weekly dosing schedule (Q2W), and if the subject achieves a complete response or a stringent complete response by cycle 12 of treatment or later, then subcutaneously administering the BCMAxCD3 bispecific antibody on a monthly dosing schedule (Q4W).

In certain embodiments, the method comprises subcutaneously administering the BCMAxCD3 bispecific antibody on the bi-weekly dosing schedule (Q2W) for at least six months, and then subcutaneously administering the BCMAxCD3 bispecific antibody on a monthly dosing schedule (Q4W).

In certain embodiments, the method comprises subcutaneously administering 1, 2 or 3 step-up doses of the BCMAxCD3 bispecific antibody prior to subcutaneously administering the first treatment dose.

In certain embodiments, the method comprises subcutaneously administering step-up doses of 60 μg/kg and 300 μg/kg and 1500 μg/kg of the BCMAxCD3 bispecific antibody prior to subcutaneously administering the first treatment dose.

In certain embodiments, the method comprises subcutaneously administering 1 or 2 step-up doses of the BCMAxCD3 bispecific antibody prior to subcutaneously administering the first treatment dose.

In certain embodiments, the method comprises subcutaneously administering 2 step-up doses of the BCMAxCD3 bispecific antibody prior to subcutaneously administering the first treatment dose.

In certain embodiments, the method comprises subcutaneously administering step-up doses of 60 μg/kg and 300 μg/kg of the BCMAxCD3 bispecific antibody prior to subcutaneously administering the first treatment dose.

In certain embodiments, the method comprises subcutaneously administering step-up doses of the BCMAxCD3 bispecific antibody 2-4 days apart from each other, prior to subcutaneously administering the first treatment dose.

In certain embodiments, the method comprises subcutaneously administering one or more step-up doses of the BCMAxCD3 bispecific antibody prior to subcutaneously administering the first treatment dose, wherein the first treatment dose is administered 2-4 days after the last step-up dose.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering the first treatment dose of the BCMAxCD3 bispecific antibody.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg for a period of time, and then subcutaneously administering the BCMAxCD3 bispecific antibody on the bi-weekly dosing schedule (Q2W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg for six months or more, and then subcutaneously administering the BCMAxCD3 bispecific antibody on the bi-weekly dosing schedule (Q2W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg, and after the subject has a partial response, a very good partial response, a complete response or a stringent complete response for six months or more (according to IMWG criteria), then subcutaneously administering the BCMAxCD3 bispecific antibody on the bi-weekly dosing schedule (Q2W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg, and after the subject has a complete response or a stringent complete response for six months or more (according to IMWG criteria), then subcutaneously administering the BCMAxCD3 bispecific antibody on the bi-weekly dosing schedule (Q2W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg, and after the subject has a partial response, a very good partial response, a complete response or a stringent complete response after four 28-day treatment cycles or more (according to IMWG criteria), then subcutaneously administering the BCMAxCD3 bispecific antibody on the bi-weekly dosing schedule (Q2W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, then subcutaneously administering the BCMAxCD3 bispecific antibody on a weekly dosing schedule (QW) at a treatment dose of 1500 μg/kg for a period of time, then subcutaneously administering the BCMAxCD3 bispecific antibody on the bi-weekly dosing schedule (Q2W) at a treatment dose of 1500 μg/kg for a period of time, and then subcutaneously administering the BCMAxCD3 bispecific antibody on a monthly dosing schedule (Q4W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, then subcutaneously administering the BCMAxCD3 bispecific antibody on a weekly dosing schedule (QW) at a treatment dose of 1500 μg/kg for a period of time, then subcutaneously administering the BCMAxCD3 bispecific antibody on the bi-weekly dosing schedule (Q2W) at a treatment dose of 1500 μg/kg for a period of time, and if the subject achieves at least CR (i.e., complete response or stringent complete response) by cycle 12 or later, then subcutaneously administering the BCMAxCD3 bispecific antibody on a monthly dosing schedule (Q4W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, then subcutaneously administering the BCMAxCD3 bispecific antibody on a weekly dosing schedule (QW) at a treatment dose of 1500 μg/kg for a period of time, then subcutaneously administering the BCMAxCD3 bispecific antibody on the bi-weekly dosing schedule (Q2W) at a treatment dose of 1500 μg/kg for at least 6 months, and then subcutaneously administering the BCMAxCD3 bispecific antibody on a monthly dosing schedule (Q4W) at a treatment dose of 1500 μg/kg.

In certain embodiments, the subject achieves a clinical response that is a PR, or a VGPR, or a CR or a sCR.

In certain embodiments, the subject achieves a clinical response that is a CR or a sCR.

In certain embodiments, the clinical response is maintained for at least 6 months.

In certain embodiments, the clinical response is maintained for at least 1 year.

In certain embodiments, the clinical response is maintained for at least 18 months.

In certain embodiments, the clinical response is maintained for at least 2 years.

In certain embodiments, fewer infections occur in the subject (e.g., compared to a subject that remains on QW dosing).

In certain embodiments, fewer grade ≥3 infections occur in the subject (e.g., compared to a subject that remains on QW dosing).

In certain embodiments, the method achieves a decrease in new infections over time, in a population of subjects with RRMM (e.g., compared to subjects that remain on a QW dosing schedule).

In certain embodiments, the method achieves a decrease in new grade ≥3 infections over time, in a population of subjects with RRMM RRMM (e.g., compared to subjects that remain on QW dosing schedule).

Embodiments of the present invention also provide a method of treating multiple myeloma in a subject in need thereof, the method comprising prophylactically administering to the subject tocilizumab (toci) prior to administering a therapeutically effective amount of a BCMAxCD3 bispecific antibody (e.g., teclistamab), wherein the method is effective in reducing the risk of cytokine release syndrome (CRS).

Embodiments of the present invention also provide methods for restarting therapy with a BCMAxCD3 bispecific antibody after a dose delay.

All methods described herein, however expressed, may be described as corresponding uses, in particular medical uses.

### BRIEF DESCRIPTION OF THE FIGURES

The following figures form part of the present specification and are included to further demonstrate certain aspects of the present invention. The invention may be better understood by reference to one or more of these figures in combination with the description of specific embodiments presented herein.

FIG. 1 illustrates a summary of the MajesTEC-1 Clinical Study Design.

FIG. 2 illustrates a summary of the MajesTEC-1 dosing schedule.

FIG. 3 provides patient characteristics of the MajesTEC-1 Q2W cohort.

FIG. 4 provides infection rates from the MajesTEC-1 Q2W cohort.

### DETAILED DESCRIPTION OF THE INVENTION

The disclosed methods can be understood more readily by reference to the following detailed description. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications and publications cited herein are incorporated by reference as if set fourth fully herein.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

“About” when used in reference to numerical ranges, cutoffs, or specific values means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of an assay, result or embodiment, “about” means within one standard deviation per the practice in the art, or a range of up to 5%, whichever is larger.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions can be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins can be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (2), based on the amino acid sequences of their constant domains.

“Antigen binding fragment” or “antigen binding domain” refers to a portion of an immunoglobulin molecule that binds an antigen. Antigen binding fragments can be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains can be linked together via a synthetic linker to form various types of single chain antibody designs where the VH/VL domains can pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“BCMA” refers to human B-cell maturation antigen, also known as CD269 or TNFRSF17 (UniProt Q02223). The extracellular domain of BCMA encompasses residues 1-54 of Q02223. Human BCMA comprises the amino acid sequence of SEQ ID NO: 1.

SEQ ID NO: 1
MLQMAGQCSQNEYFDSLLHACIPCQLRCSSNTPPLTCQRYCNASVTNSV
KGTNAILWTCLGLSLIISLAVFVLMFLLRKINSEPLKDEFKNTGSGLLGM
ANIDLEKSRTGDEIILPRGLEYTVEECTCEDCIKSKPKVDSDHCFPLPAM
EEGATILVTTKTNDYCKSLPAALSATEIEKSISAR

“Bispecific” refers to an antibody that specifically binds two distinct antigens or two distinct epitopes within the same antigen. The bispecific antibody can have cross-reactivity to other related antigens, for example to the same antigen from other species (homologs), such as human or monkey, for example Macaca cynomolgus (cynomolgus, cyno) or Pan troglodytes, or can bind an epitope that is shared between two or more distinct antigens.

“BCMAxCD3 bispecific antibody” refers to a bispecific antibody that specifically binds BCMA and CD3.

“Cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” can include a tumor.

“CD3” refers to a human antigen which is expressed on T cells as part of the multimolecular T cell receptor (TCR) complex and which consists of a homodimer or heterodimer formed from the association of two or four receptor chains: CD3 epsilon, CD3 delta, CD3 zeta and CD3 gamma. Human CD3 epsilon comprises the amino acid sequence of SEQ ID NO: 2. SEQ ID NO: 3 shows the extracellular domain of CD3 epsilon.

SEQ ID NO: 2
MQSGTHWRVLGLCLLSVGVWGQDGNEEMGGITQTPYKVSISGTTVILTC
PQYPGSEILWQHNDKNIGGDEDDKNIGSDEDHLSLKEFSELEQSGYYVCY
PRGSKPEDANFYLYLRARVCENCMEMDVMSVATIVIVDICITGGLLLLVY
YWSKNRKAKAKPVTRGAGAGGRQRGQNKERPPPVPNPDYEPIRKGQRD
LYSGLNQRRI
SEQ ID NO: 3
DGNEEMGGITQTPYKVSISGTTVILTCPQYPGSEILWQHNDKNIGGDEDD
KNIGSDEDHLSLKEFSELEQSGYYVCYPRGSKPEDANFYLYLRARVCENC
MEMD

“CH3 region” or “CH3 domain” refers to the CH3 region of an immunoglobulin. The CH3 region of human IgG1 antibody corresponds to amino acid residues 341-446. However, the CH3 region can also be any of the other antibody isotypes as described herein.

“Combination” means that two or more therapeutics are administered to a subject together in a mixture, concurrently as single agents or sequentially as single agents in any order.

“Complementarity determining regions” (CDR) are antibody regions that bind an antigen. CDRs can be defined using various delineations such as Kabat (Wu et al. J Exp Med 132: 211-50, 1970) (Kabat et al., Sequences of Proteins of Immunological Interest, 5th Ed. Public Health Service, National Institutes of Health, Bethesda, Md., 1991), Chothia (Chothia et al. J Mol Biol 196: 901-17, 1987), IMGT (Lefranc et al. Dev Comp Immunol 27: 55-77, 2003) and AbM (Martin and Thornton J Bmol Biol 263: 800-15, 1996). The correspondence between the various delineations and variable region numbering are described (see e.g. Lefranc et al. Dev Comp Immunol 27: 55-77, 2003; Honegger and Pluckthun, J Mol Biol 309:657-70, 2001; International ImMunoGeneTics (IMGT) database; Web resources, http://www_imgt_org). Available programs such as abYsis by UCL Business PLC can be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification. Preferably, the term “CDR”, “HCDR1”, “HCDR2”, “HCDR3”, “LCDR1”, “LCDR2” and “LCDR3” as used herein includes CDRs defined by the method of Kabat.

“Comprising” is intended to include examples encompassed by the terms “consisting essentially of” and “consisting of”; similarly, the term “consisting essentially of” is intended to include examples encompassed by the term “consisting of.” Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

“Fc gamma receptor” (FcγR) refers to well-known FcγRI, FcγRIIa, FcγRIIb or FcγRIII. Activating FcγR includes FcγRI, FcγRIIa and FcγRIII.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” can contain consensus framework sequences derived from human framework sequence analyses, for example as described in Knappik et al., (2000) J Mol Biol 296:57-86, or synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody can include substitutions in the frameworks so that the frameworks can not be exact copies of expressed human immunoglobulin or human immunoglobulin germline gene sequences.

“Identity” refers to a relationship between the sequences of two or more polypeptide molecules or two or more nucleic acid molecules, as determined by aligning and comparing the sequences. “Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

“Isolated” refers to a homogenous population of molecules (such as synthetic polynucleotides or a protein such as an antibody) which have been substantially separated and/or purified away from other components of the system the molecules are produced in, such as a recombinant cell, as well as a protein that has been subjected to at least one purification or isolation step. “Isolated antibody” refers to an antibody that is substantially free of other cellular material and/or chemicals and encompasses antibodies that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies can have heterogeneous glycosylation within the antibody population. Monoclonal antibody can be monospecific or multispecific such as bispecific, monovalent, bivalent or multivalent.

“Mutation” refers to an engineered or naturally occurring alteration in a polypeptide or polynucleotide sequence when compared to a reference sequence. The alteration can be a substitution, insertion or deletion of one or more amino acids or polynucleotides.

“Negative minimal residual disease status” or “negative MRD status” or “MRD negative” refers to the PerMillionCount (i.e., a point estimate of malignant myeloma cells per million nucleated cells) in a patients on-study bone marrow sample relative to their reference bone marrow sample (i.e., Teclistamab treatment naïve bone marrow sample). Based on this PerMillionCount, each sample is determined to be positive or negative. Samples are positive if the PerMillionCount is greater than or equal to the limit of sensitivity, otherwise they are negative. Negative minimal residual disease status can be determined at a sensitivity of 0.01% (10⁻⁴), 0.001% (10⁻⁵) or 0.0001% (10⁻⁶). Negative minimal residual disease status was determined using next generation sequencing (NGS).

“Pharmaceutical composition” refers to composition that comprises an active ingredient and a pharmaceutically acceptable carrier.

“Pharmaceutically acceptable carrier” or “excipient” refers to an ingredient in a pharmaceutical composition, other than the active ingredient, which is nontoxic to a subject.

“Recombinant” refers to DNA, antibodies and other proteins that are prepared, expressed, created or isolated by recombinant means when segments from different sources are joined to produce recombinant DNA, antibodies or proteins.

“Refractory” refers to a cancer that is not amendable to surgical intervention and is initially unresponsive to therapy.

“Relapsed” refers to a cancer that responded to treatment but then returns.

“Step-up dose”-refers to a dose of an active agent that is administered to a subject prior to a treatment dose. A step-up dose is lower than the treatment dose. To prevent or lessen certain toxicities, such as cytokine release syndrome (CRS), a “priming” dose strategy may include one or more lower step-up dose(s) followed by higher treatment doses.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. Except when noted, the terms “patient” or “subject” are used interchangeably.

“T cell redirecting therapeutic” refers to a molecule containing two or more binding regions, wherein one of the binding regions specifically binds a cell surface antigen on a target cell or tissue and wherein a second binding region of the molecule specifically binds a T cell antigen. Examples of cell surface antigen include a tumor associated antigen, such as BCMA. Examples of T cell antigen include, e.g., CD3. This dual/multi-target binding ability recruits T cells to the target cell or tissue leading to the eradication of the target cell or tissue.

“Therapeutically effective amount” refers to an amount effective, at doses and for periods of time necessary, to achieve a desired therapeutic result. A therapeutically effective amount can vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient.

“Treat” or “treatment” refers to both therapeutic treatment and prophylactic or preventative measures, wherein the object is to prevent or slow down (lessen) an undesired physiological change or disorder. Beneficial or desired clinical results include alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if a subject was not receiving treatment. Those in need of treatment include those already with the condition or disorder as well as those prone to have the condition or disorder or those in which the condition or disorder is to be prevented.

“Treatment dose”—refers to a dose of the active agent that is administered to a subject to treat a disease. A treatment dose may be administered at a regular dosing interval on a repetitive basis (e.g. weekly, biweekly, monthly). A treatment dose may be preceded by one or more step-up doses.

A patient that is “triple-class exposed” refers to a multiple myeloma patient that has previously been treated with (at a minimum) a proteasome inhibitor, an immunomodulatory agent and an anti-CD38 monoclonal antibody.

“Tumor cell” or a “cancer cell” refers to a cancerous, pre-cancerous or transformed cell, either in vivo, ex vivo, or in tissue culture, that has spontaneous or induced phenotypic changes. These changes do not necessarily involve the uptake of new genetic material. Although transformation can arise from infection with a transforming virus and incorporation of new genomic nucleic acid, uptake of exogenous nucleic acid or it can also arise spontaneously or following exposure to a carcinogen, thereby mutating an endogenous gene. Transformation/cancer is exemplified by morphological changes, immortalization of cells, aberrant growth control, foci formation, proliferation, malignancy, modulation of tumor specific marker levels, invasiveness, tumor growth in suitable animal hosts such as nude mice, and the like, in vitro, in vivo, and ex vivo.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated. Antibody constant chain numbering can be found for example at ImMunoGeneTics website, at IMGT Web resources at IMGT Scientific charts.

Conventional one and three-letter amino acid codes are used herein as shown in Table 1.

TABLE 1

| Amino acid | Three-letter code | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### BCMAxCD3 Bispecific Antibodies and Uses Thereof

It is well-known in the art that drug development is an unpredictable field. The lack of predictability in the art is evidenced, for example, by health authority requirements (such as those of the Food and Drug Administration) to establish a safe and effective dosing regimen for each individual drug candidate in clinical trials. Over the last decade (2011-2020), only 7.9% of all developmental drug candidates achieved FDA approval from a Phase I clinical study. See Clinical Development Success Rates and Contributing Factors 2011-2020. The rate of success is even lower in oncology, such that only 5.3% of oncology drug candidates succeed.

In the field of oncology, even for a drug that already has an established dose in a particular indication, the Food and Drug Administration (FDA) recommends further clinical studies to identify an optimal dose for a new indication; otherwise, patients may be exposed to unreasonable and significant risk, among other potential deficiencies. See, e.g., Optimizing the Dosage of Human Prescription Drugs and Biological Products for the Treatment of Oncologic Diseases; Draft Guidance for Industry; January 2023).

In patients with relapsed or refractory disease who have exhausted several therapies, multiple myeloma remains an incurable malignancy and an unmet medical need with significant morbidity and mortality. The present inventors have developed novel dosing regimens for BCMAxCD3 bispecific antibodies that provide improved safety profiles over currently approved regimens while maintaining deep and durable efficacy.

Teclistamab (also known as TECVAYLI®) is the first BCMA-directed bispecific antibody approved for the treatment of patients with relapsed or refractory multiple myeloma. Teclistamab is a bispecific B-cell maturation antigen (BCMA)-directed CD3 T-cell engager indicated for the treatment of adult patients with relapsed or refractory multiple myeloma who have received at least three or four prior lines of therapy, including a proteasome inhibitor, an immunomodulatory agent and an anti-CD38 monoclonal antibody. The efficacy of teclistamab is continuing to be evaluated in patients with relapsed or refractory multiple myeloma in a multi-center clinical study (MajesTEC-1, NCT03145181 [Phase 1] and NCT04557098 [Phase 2]). The study has included patients who had previously received at least three prior therapies, including a proteasome inhibitor, an immunomodulatory agent, and an anti-CD38 monoclonal antibody. In the MajesTEC-1 study, at 14.1-month median follow-up, teclistamab demonstrated rapid, deep and durable responses, with an overall response rate (ORR) of 63% and median progression-free survival (mPFS) of 11.3 months (see, e.g., Usmani S Z, et al. Lancet 2021; 398:665-674, and Moreau P, et al. New Engl J Med 2022; 387:495-505, which are incorporated by reference herein).

Patients with relapsed or refractory multiple myeloma (RRMM) are already at increased risk of infection, and bispecific antibodies targeting B-cell maturation antigen (BCMA) may contribute to increased infection risk, due to on-target, off-tumor toxicity. In the MajesTEC-1 clinical study, patients (N=165) received subcutaneous teclistamab 1.5 mg/kg weekly following a step-up dosing schedule (0.06 mg/kg and 0.3 mg/kg, each separated by 2-4 days). At a median follow-up of 21.6 months (range 0.26-32.69), infections were reported in 129 patients (78.2%). Overall, grade 3/4 infections occurred in 86 patients (52.1%), most commonly pneumonia (20.6%), COVID-19 (18.8%), sepsis (6.1%), and urinary tract infection (6.1%). Grade 3/4 neutropenia occurred in 65.5% of patients. Thus, there remains a need for optimized dosing regimens of BCMAxCD3 bispecific antibodies with improved safety profiles. The inventors have developed optimized dosing regimens described herein that reduce infection rates in patients, while still achieving rapid, deep and durable clinical responses.

### Antibodies of the Present Invention

Any suitable BCMAxCD3 bispecific antibody known to those skilled in the art in view of the present disclosure can be used in the invention.

Various bispecific antibody formats include formats described herein and recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule, or bispecific antibodies generated by arm exchange. Exemplary bispecific formats include dual targeting molecules include Dual Targeting (DT)-Ig (GSK/Domantis), Two-in-one Antibody (Genentech) and mAb2 (F-Star), Dual Variable Domain (DVD)-Ig (Abbott), DuoBody (Genmab), Ts2Ab (MedImmune/AZ) and BsAb (Zymogenetics), HERCULES (Biogen Idec) and TvAb (Roche), ScFv/Fc Fusions (Academic Institution), SCORPION (Emergent BioSolutions/Trubion, Zymogenetics/BMS) and Dual Affinity Retargeting Technology (Fc-DART) (MacroGenics), F (ab) 2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech), Bispecific T Cell Engager (BITE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies. Various formats of bispecific antibodies have been described, for example in Chames and Baty (2009) Curr Opin Drug Disc Dev 12:276 and in Nunez-Prado et al., (2015) Drug Discovery Today 20(5):588-594.

In some embodiments, the BCMAxCD3 bispecific antibody comprises any one of the BCMA binding domains described in WO2017/031104, the entire content of which is incorporated herein by reference. In some embodiments, the BCMAxCD3 bispecific antibody comprises any one of the CD3 binding domains described in WO2017/031104. In some embodiments, the BCMAxCD3 bispecific antibody comprises any one of the BCMAxCD3 bispecific antibodies described in WO2017/031104.

In some embodiments, the BCMAxCD3 bispecific antibody is chimeric, humanized or human.

In some embodiments, the bispecific antibody is an IgG1, an IgG2, an IgG3 or an IgG4 isotype. In preferred embodiments, the bispecific antibody is an IgG4 isotype. An exemplary wild-type IgG4 comprises an amino acid sequence of SEQ ID NO: 34.

SEQ ID NO: 34:
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRW
QEGNVFSCSVMHEALHNHYTQKSLSLSLGK

The bispecific antibody can be of any allotype. It is expected that allotype has no influence on properties of the bispecific antibodies, such as binding or Fc-mediated effector functions. Immunogenicity of therapeutic antibodies is associated with increased risk of infusion reactions and decreased duration of therapeutic response (Baert et al., (2003) N Engl J Med 348:602-08). The extent to which therapeutic antibodies induce an immune response in the host can be determined in part by the allotype of the antibody (Stickler et al., (2011) Genes and Immunity 12:213-21). Antibody allotype is related to amino acid sequence variations at specific locations in the constant region sequences of the antibody. Table 2 shows select IgG1, IgG2 and IgG4 allotypes.

TABLE 2

| | Allotype | | | | | | | |
|---|---|---|---|---|---|---|---|---|
| | Amino acid residue at position of diversity | | | | | | | |
| | (residue numbering: EU Index) | | | | | | | |
| | IgG2 | | IgG4 | | IgG1 | | | |
| | 189 | 282 | 309 | 422 | 214 | 356 | 358 | 431 |
| G2m(n) | T | M | | | | | | |
| G2m(n−) | P | V | | | | | | |
| G2m(n)/(n− | T | V | | | | | | |
| nG4m(a) | | | L | R | | | | |
| G1m(17) | | | | | K | E | M | A |
| G1m(17, 1) | | | | | K | D | L | A |

In some embodiments, the bispecific antibody comprises one or more Fc substitutions that reduces binding of the bispecific antibody to a Fcγ receptor (FcγR) and/or reduces Fc effector functions such as C1q binding, complement dependent cytotoxicity (CDC), antibody-dependent cell-mediated cytotoxicity (ADCC) or phagocytosis (ADCP). The specific substitutions can be made in comparison to the wild-type IgG4 of SEQ ID NO: 34.

Fc positions that can be substituted to reduce binding of the Fc to the activating FcγR and subsequently to reduce effector function are substitutions L234A/L235A on IgG1, V234A/G237A/P238S/H268A/V309L/A330S/P331S on IgG2, F234A/L235A on IgG4, S228P/F234A/L235A on IgG4, N297A on all Ig isotypes, V234A/G237A on IgG2, K214T/E233P/L234V/L235A/G236-deleted/A327G/P331A/D365E/L358M on IgG1, H268Q/V309L/A330S/P331S on IgG2, S267E/L328F on IgG1, L234F/L235E/D265A on IgG1, L234A/L235A/G237A/P238S/H268A/A330S/P331S on IgG1, S228P/F234A/L235A/G237A/P238S on IgG4, and S228P/F234A/L235A/G236-deleted/G237A/P238S on IgG4, wherein residue numbering is according to the EU index.

Fc substitutions that can be used to reduce CDC are a K322A substitution.

Well-known S228P substitution can further be made in IgG4 antibodies to enhance IgG4 stability.

In some embodiments, the bispecific antibody comprises one or more asymmetric substitutions in a first CH3 domain or in a second CH3 domain, or in both the first CH3 domain and the second CH3 domain.

In some embodiments, the one or more asymmetric substitutions is selected from the group consisting of F405L/K409R, wild-type/F405L_R409K, T366Y/F405A, T366W/F405W, F405W/Y407A, T394W/Y407T, T394S/Y407A, T366W/T394S, F405W/T394S and T366W/T366S_L368A_Y407V, L351Y_F405A_Y407V/T394W, T366I_K392M_T394W/F405A_Y407V, T366L_K392M_T394W/F405A_Y407V, L351Y_Y407A/T366A_K409F, L351Y_Y407A/T366V_K409F, Y407A/T366A K409F and T350V_L351Y_F405A_Y407V/T350V_T366L K392L_T394W.

In some embodiments, the BCMAxCD3 bispecific antibody is an IgG4 isotype and comprises phenylalanine at position 405 and arginine at position 409 in a first heavy chain (HC1) and leucine at position 405 and lysine at position 409 in a second heavy chain (HC2), wherein residue numbering is according to the EU Index.

In some embodiments, the BCMAxCD3 bispecific antibody further comprises proline at position 228, alanine at position 234 and alanine at position 235 in both the HC1 and the HC2.

Tables 3 and 4 provide sequences of an exemplary embodiment of a BCMAxCD3 bispecific antibody, according to the Kabat numbering system.

TABLE 3

| | | SEQ |
|---|---|---|
| | | ID |
| Region | Sequence | NO: |
| HCDR1 | SGSYFWG | 4 |
| HCDR2 | SIYYSGITYYNPSLKS | 5 |
| HCDR3 | HDGAVAGLFDY | 6 |
| LCDR1 | GGNNIGSKSVH | 7 |
| LCDR2 | DDSDRPS | 8 |
| LCDR3 | QVWDSSSDHVV | 9 |
| VH | QLQLQESGPGLVKPSETLSLTCTVSGGSISSGSY | 10 |
| | FWGWIRQPPGKGLEWIGSIYYSGITYYNPSLKS | |
| | RVTISVDTSKNQFSLKLSSVTAADTAVYYCAR | |
| | HDGAVAGLFDYWGQGTLVTVSS | |
| VL | SYVLTQPPSVSVAPGQTARITCGGNNIGSKSVH | 11 |
| | WYQQPPGQAPVVVVYDDSDRPSGIPERFSGSN | |
| | SGNTATLTISRVEAGDEAVYYCQVWDSSSDHV | |
| | VFGGGTKLTVL | |
| HC | QLQLQESGPGLVKPSETLSLTCTVSGGSISSGSY | 12 |
| | FWGWIRQPPGKGLEWIGSIYYSGITYYNPSLKS | |
| | RVTISVDTSKNQFSLKLSSVTAADTAVYYCAR | |
| | HDGAVAGLFDYWGQGTLVTVSSASTKGPSVFP | |
| | LAPCSRSTSESTAALGCLVKDYFPEPVTVSWNS | |
| | GALTSGVHTFPAVLQSSGLYSLSSVVTVPSSSL | |
| | GTKTYTCNVDHKPSNTKVDKRVESKYGPPCPP | |
| | CPAPEAAGGPSVFLFPPKPKDTLMISRTPEVTCV | |
| | VVDVSQEDPEVQFNWYVDGVEVHNAKTKPRE | |
| | EQFNSTYRVVSVLTVLHQDWLNGKEYKCKVS | |
| | NKGLPSSIEKTISKAKGQPREPQVYTLPPSQEEM | |
| | TKNQVSLTCLVKGFYPSDIAVEWESNGQPENN | |
| | YKTTPPVLDSDGSFFLYSRLTVDKSRWQEGNV | |
| | FSCSVMHEALHNHYTQKSLSLSLGK | |
| LC | SYVLTQPPSVSVAPGQTARITCGGNNIGSKSVH | 13 |
| | WYQQPPGQAPVVVVYDDSDRPSGIPERFSGSN | |
| | SGNTATLTISRVEAGDEAVYYCQVWDSSSDHV | |
| | VFGGGTKLTVLGQPKAAPSVTLFPPSSEELQAN | |
| | KATLVCLISDFYPGAVTVAWKGDSSPVKAGVE | |
| | TTTPSKQSNNKYAASSYLSLTPEQWKSHRSYSC | |
| | QVTHEGSTVEKTVAPTECS | |

TABLE 4

| | | SEQ |
|---|---|---|
| | | ID |
| Region | Sequence | NO: |
| HCDR1 | TYAMN | 14 |
| HCDR2 | RIRSKYNNYATYYAASVKG | 15 |
| HCDR3 | HGNFGNSYVSWFAY | 16 |
| LCDR1 | RSSTGAVTTSNYAN | 17 |
| LCDR2 | GTNKRAP | 18 |
| LCDR3 | ALWYSNLWV | 19 |
| VH | EVQLVESGGGLVQPGGSLRLSCAASGFTFNT | 20 |
| | YAMNWVRQAPGKGLEWVARIRSKYNNYAT | |
| | YYAASVKGRFTISRDDSKNSLYLQMNSLKTE | |
| | DTAVYYCARHGNFGNSYVSWFAYWGQGTL | |
| | VTVSS | |
| VL | QTVVTQEPSLTVSPGGTVTLTCRSSTGAVTT | 21 |
| | SNYANWVQQKPGQAPRGLIGGTNKRAPGTP | |
| | ARFSGSLLGGKAALTLSGVQPEDEAEYYCAL | |
| | WYSNLWVFGGGTKLTVLGQP | |
| HC | EVQLVESGGGLVQPGGSLRLSCAASGFTFNT | 22 |
| | YAMNWVRQAPGKGLEWVARIRSKYNNYAT | |
| | YYAASVKGRFTISRDDSKNSLYLQMNSLKTE | |
| | DTAVYYCARHGNFGNSYVSWFAYWGQGTL | |
| | VTVSSASTKGPSVFPLAPCSRSTSESTAALGC | |
| | LVKDYFPEPVTVSWNSGALTSGVHTFPAVL | |
| | QSSGLYSLSSVVTVPSSSLGTKTYTCNVDHK | |
| | PSNTKVDKRVESKYGPPCPPCPAPEAAGGPS | |
| | VFLFPPKPKDTLMISRTPEVTCVVVDVSQED | |
| | PEVQFNWYVDGVEVHNAKTKPREEQFNSTY | |
| | RVVSVLTVLHQDWLNGKEYKCKVSNKGLPS | |
| | SIEKTISKAKGQPREPQVYTLPPSQEEMTKNQ | |
| | VSLTCLVKGFYPSDIAVEWESNGQPENNYKT | |
| | TPPVLDSDGSFLLYSKLTVDKSRWQEGNVFS | |
| | CSVMHEALHNHYTQKSLSLSLGK | |
| LC | QTVVTQEPSLTVSPGGTVTLTCRSSTGAVTT | 23 |
| | SNYANWVQQKPGQAPRGLIGGTNKRAPGTP | |
| | ARFSGSLLGGKAALTLSGVQPEDEAEYYCAL | |
| | WYSNLWVFGGGTKLTVLGQPKAAPSVTLFP | |
| | PSSEELQANKATLVCLISDFYPGAVTVAWKA | |
| | DSSPVKAGVETTTPSKQSNNKYAASSYLSLT | |
| | PEQWKSHRSYSCQVTHEGSTVEKTVAPTECS | |

In some embodiments, the BCMAxCD3 bispecific antibody is CC-93269, BI 836909, JNJ-64007957 (teclistamab), or PF-06863135. In preferred embodiments, the BCMAxCD3 bispecific antibody is teclistamab (also referred to herein as Tec), which has the sequences described in Tables 3 and 4.

Teclistamab and its methods of use are described, for example, in WO 2017/031104, WO 2019/220369 and WO 2021/228783, which are incorporated by reference herein. According to particular embodiments, the BCMAxCD3 bispecific antibody has an amino acid sequence with at least 80%, at least 85%, at least 90%, at least 95%, at least 98%, or at least 99% sequence identity to the amino acid sequence of teclistamab.

Additional embodiments of BCMAxCD3 and GPRC5DxCD3 bispecific antibodies that may be used in combination regimens of the present invention are described below.

In certain embodiments, the BCMAxCD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

In certain embodiments, the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.

In certain embodiments, the BCMAxCD3 bispecific antibody is an IgG1, an IgG2, an IgG3 or an IgG4 isotype.

In certain embodiments, the BCMAxCD3 bispecific antibody is an IgG4 isotype.

In certain embodiments, the BCMAxCD3 bispecific antibody comprises one or more substitutions in its Fc region.

In certain embodiments, the BCMAxCD3 bispecific antibody is an IgG4 isotype and comprises S228P, F234A and L235A substitutions in its Fc region (according to EU numbering).

In certain embodiments, the BCMAxCD3 bispecific antibody is an IgG4 isotype and comprises S228P, F234A, L235A F405L and R409K substitutions in its Fc region (according to EU numbering).

In certain embodiments, the Fc region of the BCMA-binding arm comprises S228P, F234A and L235A substitutions in its Fc region (according to EU numbering).

In certain embodiments, the Fc region of the CD3 CD3-binding arm comprises S228P, F234A, L235A, F405L, and R409K substitutions in its Fc region (according to EU numbering).

In certain embodiments, the BCMAxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.

In certain embodiments, the BCMAxCD3 bispecific antibody comprises a first heavy chain (HC1) having at least 90% identity to the amino acid sequence of SEQ ID NO: 12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.

In certain embodiments, the BCMAxCD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.

In certain embodiments, the BCMAxCD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.

In certain embodiments, the BCMAxCD3 bispecific antibody is teclistamab.

Patient Populations with Multiple Myeloma

The BCMAxCD3 bispecific antibodies disclosed herein are for use in treating multiple myeloma in a subject, for example a human subject, wherein the subject is relapsed or refractory to treatment with one or more prior anti-cancer treatments. Relapsed disease means a cancer has come back. Refractory disease means a cancer has not improved with treatment or has stopped responding to treatment.

In some embodiments, the subject is relapsed or refractory to treatment with a therapeutic used to treat multiple myeloma or other hematological malignancies.

In certain embodiments, the subject has had been treated with from 2 to 14 prior lines of therapy.

In certain embodiments, the subject has received at least three prior lines of therapy.

In certain embodiments, the subject has received at least four prior lines of therapy.

In certain embodiments, the subject has received at least five prior lines of therapy (penta-drug exposed).

In certain embodiments, the subject has received at least three prior lines of therapy, including a proteasome inhibitor, an immunomodulatory agent and an anti-CD38 monoclonal antibody.

In certain embodiments, the subject has received at least four prior lines of therapy, including a proteasome inhibitor, an immunomodulatory agent and an anti-CD38 monoclonal antibody.

In particular embodiments, the patients are relapsed or refractory or intolerant to the last line of therapy (LOT); were exposed to a proteasome inhibitor, immunomodulatory drug, and anti-CD38 therapy; and had measurable disease.

In some embodiments, the subject has received three anti-cancer therapies prior to administration of the BCMAxCD3 bispecific antibody. In one embodiment, the three prior anti-cancer therapies are a proteasome inhibitor (PI), an immunomodulatory drug (IMiD), and an anti-CD38 antibody. In certain such embodiments, the proteasome inhibitor is bortezomib, carfilzomib or ixazomib, the immunomodulatory drug (IMiD) is lenalidomide, pomalidomide or thalidomide and the anti-CD38 antibody is daratumumab or isatuximab.

In one embodiment, the proteasome inhibitor is bortezomib, the immunomodulatory drug (IMiD) is lenalidomide and the anti-CD38 antibody is daratumumab. In one embodiment, the proteasome inhibitor is bortezomib, the immunomodulatory drug (IMiD) is lenalidomide and the anti-CD38 antibody is isatuximab. In one embodiment, the proteasome inhibitor is bortezomib, the immunomodulatory drug (IMiD) is pomalidomide and the anti-CD38 antibody is daratumumab. In one embodiment, the proteasome inhibitor is bortezomib, the immunomodulatory drug (IMiD) is pomalidomide and the anti-CD38 antibody is isatuximab. In one embodiment, the proteasome inhibitor is bortezomib, the immunomodulatory drug (IMiD) is thalidomide and the anti-CD38 antibody is daratumumab. In one embodiment, the proteasome inhibitor is bortezomib, the immunomodulatory drug (IMiD) is thalidomide and the anti-CD38 antibody is isatuximab.

In one embodiment, the proteasome inhibitor is carfilzomib, the immunomodulatory drug (IMiD) is lenalidomide and the anti-CD38 antibody is daratumumab. In one embodiment, the proteasome inhibitor is carfilzomib, the immunomodulatory drug (IMiD) is lenalidomide and the anti-CD38 antibody is isatuximab. In one embodiment, the proteasome inhibitor is carfilzomib, the immunomodulatory drug (IMiD) is pomalidomide and the anti-CD38 antibody is daratumumab. In one embodiment, the proteasome inhibitor is carfilzomib, the immunomodulatory drug (IMiD) is pomalidomide and the anti-CD38 antibody is isatuximab. In one embodiment, the proteasome inhibitor is carfilzomib, the immunomodulatory drug (IMiD) is thalidomide and the anti-CD38 antibody is daratumumab. In one embodiment, the proteasome inhibitor is carfilzomib, the immunomodulatory drug (IMiD) is thalidomide and the anti-CD38 antibody is isatuximab.

In one embodiment, the proteasome inhibitor is ixazomib, the immunomodulatory drug (IMiD) is lenalidomide and the anti-CD38 antibody is daratumumab. In one embodiment, the proteasome inhibitor is ixazomib, the immunomodulatory drug (IMiD) is lenalidomide and the anti-CD38 antibody is isatuximab. In one embodiment, the proteasome inhibitor is ixazomib, the immunomodulatory drug (IMiD) is pomalidomide and the anti-CD38 antibody is daratumumab. In one embodiment, the proteasome inhibitor is ixazomib, the immunomodulatory drug (IMiD) is pomalidomide and the anti-CD38 antibody is isatuximab. In one embodiment, the proteasome inhibitor is ixazomib, the immunomodulatory drug (IMiD) is thalidomide and the anti-CD38 antibody is daratumumab. In one embodiment, the proteasome inhibitor is ixazomib, the immunomodulatory drug (IMiD) is thalidomide and the anti-CD38 antibody is isatuximab.

In some embodiments, the subject is refractory or relapsed to treatment with one or more treatments or therapies, such as THALOMID® (thalidomide), REVLIMID® (lenalidomide), POMALYST® (pomalidomide), VELCADE® (bortezomib), NINLARO (ixazomib), KYPROLIS® (carfilzomib), FARADYK® (panobinostat), AREDIA® (pamidronate), ZOMETA® (zoledronic acid), DARZALEX® (daratumumab), elotozumab or melphalan, Xpovio® (Selinexor), Venclexta® (Venetoclax), GSK 916, CAR-T therapies, or other BCMA-directed therapies.

Various qualitative and/or quantitative methods can be used to determine relapse or refractory nature of the disease. Symptoms that can be associated are for example a decline or plateau of the well-being of the patient or re-establishment or worsening of various symptoms associated with solid tumors, and/or the spread of cancerous cells in the body from one location to other organs, tissues or cells.

In some embodiments, the multiple myeloma is relapsed or refractory to treatment with an anti-CD38 antibody, selinexor, venetoclax, lenalinomide, bortezomib, pomalidomide, carfilzomib, elotozumab, ixazomib, melphalan or thalidomide, or any combination thereof.

In one embodiment, the anti-CD38 antibody is daratumumab.

In another embodiment, the anti-CD38 antibody is isatuximab

In some embodiments, the multiple myeloma is a high-risk multiple myeloma. Subjects with high-risk multiple myeloma are known to relapse early and have poor prognosis and outcome. Subjects can be classified as having high-risk multiple myeloma is they have one or more of the following cytogenetic abnormalities: t(4;14)(p16;q32), t(14;16)(q32;q23), del17p, 1qAmp, t(4;14)(p16;q32) and t(14;16)(q32;q23), t(4;14)(p16;q32) and del17p, t(14;16)(q32;q23) and del17p, or t(4;14)(p16;q32), t(14;16)(q32;q23) and del17p. In some embodiments, the subject having the high-risk multiple myeloma has one or more chromosomal abnormalities comprising: t(4;14)(p16;q32), t(14;16)(q32;q23), del17p, 1qAmp, t(4;14)(p16;q32) and t(14;16)(q32;q23), t(4;14)(p16;q32) and del17p, t(14;16)(q32;q23) and del17p; or t(4;14)(p16;q32), t(14;16)(q32;q23) and del17p, or any combination thereof.

The cytogenetic abnormalities can be detected for example by fluorescent in situ hybridization (FISH). In chromosomal translocations, an oncogene is translocated to the IgH region on chromosome 14q32, resulting in dysregulation of these genes. t(4;14)(p16;q32) involves translocation of fibroblast growth factor receptor 3 (FGFR3) and multiple myeloma SET domain containing protein (MMSET) (also called WHSC1/NSD2), and t(14;16)(q32;q23) involves translocation of the MAF transcription factor C-MAF. Deletion of 17p (del17p) involves loss of the p53 gene locus.

Chromosomal rearrangements can be identified using well known methods, for example fluorescent in situ hybridization, karyotyping, pulsed field gel electrophoresis, or sequencing.

### Bi-Weekly and Monthly Administration of a BCMAxCD3 Bispecific Antibody

The inventors have developed novel dosing regimens for BCMAxCD3 bispecific antibodies that provide improved safety profiles over currently approved regimens while maintaining deep and durable clinical responses over time.

Teclistamab is the first B-cell maturation antigen (BCMA) bispecific antibody approved for the treatment of relapsed/refractory multiple myeloma (RRMM) at a dose of 1.5 mg/kg weekly (QW) given subcutaneously. A less frequent dosing schedule offers added convenience and flexibility to patients, physicians, and caregivers. According to certain embodiments, a dose of 1.5 mg/kg every two weeks (Q2W) given subcutaneously is safe and effective in the treatment of RRMM. According to additional embodiments, a dose of 1.5 mg/kg every four weeks (Q4W) given subcutaneously is safe and effective in the treatment of RRMM.

As used herein, “weight-based” refers to administration of a dose amount that is based on the subject's specific body weight; for example, 3 mg/kg refers to a dose of 3 milligrams of antibody per kilogram of the subject's body weight. Unless otherwise specified herein, when a dose is described in a unit of “mg/kg” or “μg/kg,” weight-based dosing is being employed.

Unless otherwise specified herein, a BCMAxCD3 bispecific antibody, such as teclistamab, is administered on a dosing schedule based on sequential 28-day cycles, for example, Cycle 1 starts on Day 1 of Cycle 1 and ends on Day 28 of Cycle 1, and then Day 1 of Cycle 2 starts the day after Day 28 of Cycle 1 and ends on Day 28 of Cycle 2, and then Day 1 of Cycle 3 starts the day after Day 28 of Cycle 2 and ends on Day 28 of Cycle 3, and so on.

As used herein “Q4W” means once every four weeks, “Q2W” (also referred to as “bi-weekly” or “biweekly”) means once every two weeks, and “QW” (also referred to as “weekly”) means once weekly. Q4W is sometimes referred to herein as “monthly” but technically refers to once every 4 weeks or once every 28 days (e.g., in 28-day cycles, a first treatment dose occurs on Day 1 of Cycle 1, a second treatment dose occurs on Day 1 of Cycle 2, etc.). Administration of a treatment dose once weekly (QW) is also referred to herein as a weekly dosing schedule. Administration of a treatment dose once every two weeks (Q2W) is also referred to herein as a bi-weekly dosing schedule. Administration of a treatment dose once every four weeks (Q4W) is also referred to herein as a monthly dosing schedule.

Additional abbreviations used herein include the following: CR, complete response; PR, partial response; Q2W, once every 2 weeks; Q4W, once every 4 weeks; QW, once weekly; RP2D, recommended phase 2 dose; SUD, step-up dose.

As used herein, the RP2D of teclistamab refers to the FDA-approved weight-based regimen for teclistamab monotherapy, based on the MajesTEC-1 clinical study, which includes treatment doses of 1.5 mg/kg teclistamab subcutaneously (SC) administered weekly until disease progression or unacceptable toxicity, wherein the treatment doses are preceded by step-up doses of 0.06 and 0.3 mg/kg. In accordance with embodiments of the present invention, subjects in MajesTEC-1 could switch from weekly to Q2W and subsequently to Q4W dosing as described herein.

According to embodiments of the present invention, methods of treating multiple myeloma are effective in eliciting a clinical response in a subject as determined by International Myeloma Working Group (IMWG) response criteria. According to particular embodiments, the methods of treatment are effective in eliciting a partial response, a very good partial response, a complete response or a stringent complete response, as determined by IMWG response criteria. As used herein, overall response rate (ORR) refers to the percentage of patients in a population that achieve a partial response (PR) or better, i.e., a partial response, very good partial response, complete response or stringent complete response. IMWG criteria for response to Multiple 5 Myeloma treatment are provided in Table A below.

TABLE A

| Response | Response Criteria |
|---|---|
| sCR = stringent | □ CR as defined below, plus |
| complete | □ Normal FLC ratio, and |
| response | □ Absence of clonal PCs by immunohistochemistry, |
| | immunofluorescence^{a} or 2- to 4-color flow cytometry |
| CR = complete | □ Negative immunofixation on the serum and urine, |
| response | and |
| | □ Disappearance of any soft tissue plasmacytomas, |
| | and |
| | □ <5% PCs in bone marrow |
| VGPR = very | □ Serum and urine M-component detectable by |
| good partial | immunofixation but not on electrophoresis, or |
| response | □ ≥90% reduction in serum M-protein plus urine |
| | M-protein <100 mg/24 hours |
| PR = partial | □ ≥50% reduction of serum M-protein and reduction |
| response | in 24-hour urinary M-protein by ≥90% or to <200 |
| | mg/24 hours |
| | □ If the serum and urine M-protein are not |
| | measurable, a decrease of ≥50% in the difference |
| | between involved and uninvolved FLC levels is |
| | required in place of the M-protein criteria |
| | □ If serum and urine M-protein are not |
| | measurable, and serum free light assay is also not |
| | measurable, ≥50% reduction in bone marrow PCs is |
| | required in place of M-protein, |
| | provided baseline bone marrow plasma cell |
| | percentage was ≥30% |
| | □ In addition to the above criteria, if present at |
| | baseline, a ≥50% reduction in the size of soft tissue |
| | plasmacytomas is also required. |

CR = complete response;
FLC = free light chain;
IMWG = International Myeloma Working Group;
M-protein = monoclonal paraprotein;
MR = minimal response;
PC = plasma cell;
PD = progressive disease;
PR = partial response;
sCR = stringent complete response;
SD = stable disease;
VGPR = very good partial response
^{a}Presence/absence of clonal cells is based upon the kappa/lambda ratio. An abnormal kappa/lambda ratio by immunohistochemistry or immunofluorescence requires a minimum of 100 plasma cells for analysis. An abnormal ratio reflecting presence of an abnormal clone is kappa/lambda of >4:1 or <1:2.
* Clarifications to IMWG criteria for coding CR and VGPR in subjects in whom the only measurable disease is by serum FLC levels: CR in such subjects indicates a normal FLC ratio of 0.26 to 1.65 in addition to CR criteria listed above. VGPR in such subjects requires a >90% decrease in the difference between involved and uninvolved FLC levels.

IMWG criteria for response to Multiple Myeloma treatment are also described, for example, in Durie et al., Kumar et al. and Rajkumar et al., which are incorporated by reference herein: Durie B G, Harousseau J L, Miguel J S, et al. International uniform response criteria for multiple myeloma. Leukemia. 2006; 20(9):1467-1473; Kumar S, Paiva B, Anderson K C, et al. International Myeloma Working Group consensus criteria for response and minimal residual disease assessment in multiple myeloma. Lancet Oncol. 2016; 17(8):e328-346; Rajkumar S V, Harousseau J L, Durie B, et al. Consensus recommendations for the uniform reporting of clinical Trials: report of the International Myeloma Workshop Consensus Panel 1. Blood. 2011; 117(18):4691-4695.

Study 64007957MMY1001 (MajesTEC-1) is a single-arm, open-label, multicenter study of teclistamab administered as monotherapy to adult subjects with relapsed or refractory multiple myeloma. Following identification of the proposed RP2D in Phase 1, the anti-myeloma activity and safety of teclistamab were further evaluated in Phase 2 in cohorts of subjects with relapsed or refractory multiple myeloma with unmet medical need. The pivotal population included in the All Treated Analysis Set of MajesTEC-1 comprises 165 subjects (40 treated in Phase 1 and 125 treated in Cohort A of Phase 2).

According to a weekly dosing schedule in MajesTEC-1, teclistamab is administered subcutaneously according to the following weight-based dosing schedule shown in Table B, wherein mg/kg refers to mg of teclistamab per kg of the patient's body weight:

TABLE B

| | | |
|---|---|---|
| Dosing | | |
| Schedule | Day | Dose |

| | | | |
|---|---|---|---|
| Step-up | Day 1 | Step-up | 0.06 mg/kg |
| Dosing | | dose 1 | |
| Schedule | Day 4 ^{b} | Step-up | 0.3 mg/kg |
| | | dose 2 | |
| | Day 7 ^{c} | First | 1.5 mg/kg |
| | | treatment | |
| | | dose | |
| Weekly | One week after | Subsequent | 1.5 mg/kg |
| Dosing | first treatment dose | treatment | once weekly |
| Schedule | and weekly | doses | |
| | thereafter | | |

^{b} Step-up dose 2 may be given between 2 to 4 days after step-up dose 1 and may be given up to 7 days after step-up dose 1 to allow for resolution of adverse reactions.
^{c} First treatment dose may be given between 2 to 4 days after step-up dose 2 and may be given up to 7 days after step-up dose 2 to allow for resolution of adverse reactions.

According to certain embodiments, patients from the MajesTEC-1 study who transitioned from QW to less frequent Q2W dosing of teclistamab had sustained remission, with a median duration of response of 20.5 months from the date of switch.

According to certain embodiments, a subject begins treatment with teclistamab on a weekly schedule and subsequently switches to a bi-weekly dosing schedule after a 10 period of time. According to an embodiment, a method of treatment comprises administering the BCMAxCD3 bispecific antibody according to the following dosing schedule shown in Table C:

TABLE C

| Dosing | | | |
|---|---|---|---|
| Schedule | Day | Dose | |
| Step-up | Day 1 | Step-up dose 1 | 0.06 mg/kg |
| Dosing | Day 4 | Step-up dose 2 | 0.3 mg/kg |
| Schedule | Day 7 | First treatment dose | 1.5 mg/kg |
| Weekly | One | Subsequent treatment | 1.5 mg/kg once |
| Dosing | week after first | doses | every two weeks |
| Schedule | treatment dose | | |
| | and once every | | |
| | two weeks | | |
| | thereafter | | |

According to certain embodiments, the BCMAxCD3 bispecific antibody is administered according to the following schedule shown in TABLE D:

TABLE D

| | | |
|---|---|---|
| Dosing | | |
| Schedule | Day | Dose |

All Patients

| | | | |
|---|---|---|---|
| Step-up | Day 1 | Step-up dose 1 | 0.06 mg/kg |
| Dosing | Day 4^{a} | Step-up dose 2 | 0.3 mg/kg |
| Schedule | Day 7^{b} | First treatment dose | 1.5 mg/kg |
| Weekly | One week | Subsequent treatment | 1.5 mg/kg once |
| Dosing | after first | doses | weekly |
| Schedule | treatment | | |
| | dose and | | |
| | weekly | | |
| | thereafter | | |

Patients who have a response for a minimum of 6 months

| Biweekly | Consider reducing the dosing frequency to 1.5 |
|---|---|
| (every two | mg/kg every two weeks |
| weeks) | |
| dosing | |
| schedule | |

^{a}Step-up dose 2 may be given between 2 to 4 days after step-up dose 1 and may be given up to 7 days after step-up dose 1 to allow for resolution of adverse reactions.
^{b}First treatment dose may be given between 2 to 4 days after step-up dose 2 and may be given up to 7 days after step-up dose 2 to allow for resolution of adverse reactions.

According to certain embodiments, the BCMAxCD3 bispecific antibody is administered according to the following schedule shown in TABLE E:

TABLE E

| | | |
|---|---|---|
| Dosing | | |
| schedule | Day | Dose^{a} |

All patients

| | | | |
|---|---|---|---|
| Step-up dosing | Day 1 | Step-up dose 1 | 0.06 mg/kg SC |
| schedule^{b} | | | single dose |
| | Day 3^{c} | Step-up dose 2 | 0.3 mg/kg SC |
| | | | single dose |
| | Day 5^{d} | First | 1.5 mg/kg SC |
| | | maintenance | single dose |
| | | dose | |
| Weekly dosing | One week after | Subsequent | 1.5 mg/kg SC |
| schedule^{b} | first maintenance | maintenance | once weekly |
| | dose and weekly | doses | |
| | thereafter^{e} | | |

Patients who have a complete response or better for a
minimum of 6 months

| Biweekly (every | Consider reducing the dosing frequency to |
|---|---|
| two weeks) | 1.5 mg/kg SC every two weeks |
| dosing schedulee | |

^{a}Dose is based on actual body weight and should be administered subcutaneously.
^{b}See Table 7 for recommendations on restarting Teclistamab after dose delays.
^{c}Step-up dose 2 may be given between two to seven days after Step-up dose 1.
^{d}First maintenance dose may be given between two to seven days after Step-up dose 2. This is the first full maintenance dose (1.5 mg/kg).
^{e}Maintain a minimum of five days between weekly maintenance doses.

According to certain embodiments, the BCMAxCD3 bispecific antibody is administered according to the following schedule shown in TABLE F:

TABLE F

| | | |
|---|---|---|
| Dosing | | |
| Schedule | Day | Dose |

All Patients

| | | | |
|---|---|---|---|
| Step-up | Day 1 | Step-up dose 1 | 0.06 mg/kg |
| Dosing | Day 4^{a} | Step-up dose 2 | 0.3 mg/kg |
| Schedule | Day 7^{b} | First treatment dose | 1.5 mg/kg |
| Weekly | One week | Subsequent | 1.5 mg/kg |
| Dosing | after first | treatment doses | once weekly |
| Schedule | treatment | | |
| | dose and | | |
| | weekly | | |
| | thereafter | | |

Patients who have achieved and maintained a complete
response or better for aminimum of 6 months

| | |
|---|---|
| Biweekly | The dosing frequency may be decreased to 1.5 |
| (every two | mg/kg every two weeks |

weeks)
dosing
schedule

^{a}Step-up dose 2 may be given between 2 to 4 days after step-up dose 1 and may be given up to 7 days after step-up dose 1 to allow for resolution of adverse reactions.
^{b}First treatment dose may be given between 2 to 4 days after step-up dose 2 and may be given up to 7 days after step-up dose 2 to allow for resolution of adverse reactions.

According to an embodiment, a teclistamab dosing regimen comprises step-up doses of 0.06 mg/kg and 0.3 mg/kg followed by 1.5 mg/kg once weekly until disease progression or unacceptable toxicity; and, in patients who have a response for a minimum of 6 months, optionally reducing dosing frequency to 1.5 mg/kg every two weeks until disease progression or unacceptable toxicity.

According to an embodiment, a teclistamab dosing regimen comprises step-up doses of 0.06 mg/kg and 0.3 mg/kg followed by 1.5 mg/kg once weekly until disease progression or unacceptable toxicity; and, in patients that have a complete response or better for a minimum of 6 months, optionally reducing dosing frequency to 1.5 mg/kg every two weeks until disease progression or unacceptable toxicity.

Additional embodiments of dosing regimens of the present invention are described below.

In certain embodiments, a method of treating multiple myeloma in a subject in need thereof comprises administering to the subject treatment doses of a BCMAxCD3 bispecific antibody on a bi-weekly dosing schedule (Q2W).

In certain embodiments, the method comprises subcutaneously administering to the subject one or more step-up doses of the BCMAxCD3 bispecific antibody prior to administering the first treatment dose of the BCMAxCD3 bispecific antibody.

In certain embodiments, the method comprises subcutaneously administering the BCMAxCD3 bispecific antibody on the bi-weekly dosing schedule (Q2W) at a treatment dose of from about 1500 μg/kg to about 3000 μg/kg.

In certain embodiments, the method comprises subcutaneously administering the BCMAxCD3 bispecific antibody on the bi-weekly dosing schedule (Q2W) at a treatment dose of about 1500 μg/kg.

In certain embodiments, the method comprises subcutaneously administering the BCMAxCD3 bispecific antibody on the bi-weekly dosing schedule (Q2W) at a treatment dose of about 3000 μg/kg.

In certain embodiments, the method comprises subcutaneously administering to the subject at least one treatment dose of the BCMAxCD3 bispecific antibody on a weekly dosing schedule (QW) and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) and, if the subject has a complete response or better (i.e., a complete response or stringent complete response according to IMWG 2016 criteria) for a minimum of 6 months, then administering to the subject treatment doses of the BCMAxCD3 bispecific antibody on the bi-weekly dosing schedule (Q2W) (i.e., reducing the frequency of the treatment doses from weekly to bi-weekly after the subject exhibits a complete response or better (complete response or stringent response) for a minimum of 6 months.

In certain embodiments, the method comprises subcutaneously administering treatment doses of the BCMAxCD3 bispecific antibody on a weekly dosing schedule (QW) for at least one 28-day cycle of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least two 28-day cycles of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least three 28-day cycles of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least four 28-day cycles of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least five 28-day cycles of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least six 28-day cycles of treatment, and subsequently administering treatment doses of the BCMAxCD3 bispecific antibody on the bi-weekly dosing schedule (Q2W).

In certain embodiments, the method comprises subcutaneously administering treatment doses of the BCMAxCD3 bispecific antibody on a weekly dosing schedule (QW) for at least six months, and subsequently administering treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a partial response, a very good partial response, a complete response or a stringent complete response, as determined by IMWG response criteria, and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a very good partial response, a complete response or a stringent complete response, as determined by IMWG response criteria, and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a complete response or a stringent complete response, as determined by IMWG response criteria, and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a partial response, a very good partial response, a complete response or a stringent complete response after four or more cycles of treatment, as determined by IMWG response criteria, and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a partial response, a very good partial response, a complete response or a stringent complete response for six or more months, as determined by IMWG response criteria, and subsequently administering to the subject treatment doses of the BCMAxCD3 bispecific antibody on the bi-weekly dosing schedule (Q2W).

In certain embodiments, the method comprises subcutaneously administering to the subject treatment doses of the BCMAxCD3 bispecific antibody on a weekly dosing schedule (QW) until the subject achieves a complete response or a stringent complete response for six or more months, as determined by IMWG response criteria, and subsequently administering to the subject treatment doses of the BCMAxCD3 bispecific antibody on the bi-weekly dosing schedule (Q2W).

In certain embodiments, each treatment dose amount of the BCMAxCD3 bispecific antibody administered on the weekly dosing schedule (QW) is the same treatment dose amount of the BCMAxCD3 bispecific antibody administered on the bi-weekly dosing schedule (Q2W).

In certain embodiments, the method comprises subcutaneously administering the BCMAxCD3 bispecific antibody on the weekly dosing schedule (QW) at a treatment dose of from about 1500 μg/kg to about 3000 μg/kg.

In certain embodiments, the method comprises subcutaneously administering the BCMAxCD3 bispecific antibody on the weekly dosing schedule (QW) at a treatment dose of about 1500 μg/kg.

In certain embodiments, the method comprises subcutaneously administering the BCMAxCD3 bispecific antibody on the weekly dosing schedule (QW) at a treatment dose of about 3000 μg/kg.

In certain embodiments, the method comprises subcutaneously administering the BCMAxCD3 bispecific antibody on the weekly dosing schedule (QW) at a treatment dose of about 1500 μg/kg, and then subcutaneously administering the BCMAxCD3 bispecific antibody on the bi-weekly dosing schedule (Q2W) at a treatment dose of about 1500 μg/kg.

In certain embodiments, the method comprises subcutaneously administering the BCMAxCD3 bispecific antibody on the weekly dosing schedule (QW) at a treatment dose of about 1500 μg/kg, and then subcutaneously administering the BCMAxCD3 bispecific antibody on the bi-weekly dosing schedule (Q2W) at a treatment dose of about 3000 μg/kg.

In certain embodiments, the method comprises subcutaneously administering the BCMAxCD3 bispecific antibody on the bi-weekly dosing schedule (Q2W), and then subcutaneously administering the BCMAxCD3 bispecific antibody on a monthly dosing schedule (Q4W).

In certain embodiments, the method comprises subcutaneously administering the BCMAxCD3 bispecific antibody on a weekly dosing schedule (QW), and then subcutaneously administering the BCMAxCD3 bispecific antibody on the bi-weekly dosing schedule (Q2W), and then subcutaneously administering the BCMAxCD3 bispecific antibody on a monthly dosing schedule (Q4W).

In certain embodiments, the method comprises subcutaneously administering the BCMAxCD3 bispecific antibody on the bi-weekly dosing schedule (Q2W), and if the subject achieves a partial response, a very good partial response, a complete response or a stringent complete response, then subcutaneously administering the BCMAxCD3 bispecific antibody on a monthly dosing schedule (Q4W).

In certain embodiments, the method comprises subcutaneously administering the BCMAxCD3 bispecific antibody on the bi-weekly dosing schedule (Q2W), and if the subject achieves a complete response or a stringent complete response, then subcutaneously administering the BCMAxCD3 bispecific antibody on a monthly dosing schedule (Q4W).

In certain embodiments, the method comprises subcutaneously administering the BCMAxCD3 bispecific antibody on the bi-weekly dosing schedule (Q2W), and if the subject achieves a complete response or a stringent complete response by cycle 12 of treatment or later, then subcutaneously administering the BCMAxCD3 bispecific antibody on a monthly dosing schedule (Q4W).

In certain embodiments, the method comprises subcutaneously administering the BCMAxCD3 bispecific antibody on the bi-weekly dosing schedule (Q2W) for at least six months, and then subcutaneously administering the BCMAxCD3 bispecific antibody on a monthly dosing schedule (Q4W).

In certain embodiments, the method comprises subcutaneously administering 1, 2 or 3 step-up doses of the BCMAxCD3 bispecific antibody prior to subcutaneously administering the first treatment dose.

In certain embodiments, the method comprises subcutaneously administering step-up doses of 60 μg/kg and 300 μg/kg and 1500 μg/kg of the BCMAxCD3 bispecific antibody prior to subcutaneously administering the first treatment dose.

In certain embodiments, the method comprises subcutaneously administering 1 or 2 step-up doses of the BCMAxCD3 bispecific antibody prior to subcutaneously administering the first treatment dose.

In certain embodiments, the method comprises subcutaneously administering 2 step-up doses of the BCMAxCD3 bispecific antibody prior to subcutaneously administering the first treatment dose.

In certain embodiments, the method comprises subcutaneously administering step-up doses of 60 μg/kg and 300 μg/kg of the BCMAxCD3 bispecific antibody prior to subcutaneously administering the first treatment dose.

In certain embodiments, the method comprises subcutaneously administering step-up doses of the BCMAxCD3 bispecific antibody 2-4 days apart from each other, prior to subcutaneously administering the first treatment dose.

In certain embodiments, the method comprises subcutaneously administering one or more step-up doses of the BCMAxCD3 bispecific antibody prior to subcutaneously administering the first treatment dose, wherein the first treatment dose is administered 2-4 days after the last step-up dose.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering the first treatment dose of the BCMAxCD3 bispecific antibody.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg for a period of time, and then subcutaneously administering the BCMAxCD3 bispecific antibody on the bi-weekly dosing schedule (Q2W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg for six months or more, and then subcutaneously administering the BCMAxCD3 bispecific antibody on the bi-weekly dosing schedule (Q2W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg, and after the subject has a partial response, a very good partial response, a complete response or a stringent complete response for six months or more (according to IMWG criteria), then subcutaneously administering the BCMAxCD3 bispecific antibody on the bi-weekly dosing schedule (Q2W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg, and after the subject has a complete response or a stringent complete response for six months or more (according to IMWG criteria), then subcutaneously administering the BCMAxCD3 bispecific antibody on the bi-weekly dosing schedule (Q2W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg, and after the subject has a partial response, a very good partial response, a complete response or a stringent complete response after four 28-day treatment cycles or more (according to IMWG criteria), then subcutaneously administering the BCMAxCD3 bispecific antibody on the bi-weekly dosing schedule (Q2W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, then subcutaneously administering the BCMAxCD3 bispecific antibody on a weekly dosing schedule (QW) at a treatment dose of 1500 μg/kg for a period of time, then subcutaneously administering the BCMAxCD3 bispecific antibody on the bi-weekly dosing schedule (Q2W) at a treatment dose of 1500 μg/kg for a period of time, and then subcutaneously administering the BCMAxCD3 bispecific antibody on a monthly dosing schedule (Q4W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, then subcutaneously administering the BCMAxCD3 bispecific antibody on a weekly dosing schedule (QW) at a treatment dose of 1500 μg/kg for a period of time, then subcutaneously administering the BCMAxCD3 bispecific antibody on the bi-weekly dosing schedule (Q2W) at a treatment dose of 1500 μg/kg for a period of time, and if the subject achieves at least CR (i.e., complete response or stringent complete response) by cycle 12 or later, then subcutaneously administering the BCMAxCD3 bispecific antibody on a monthly dosing schedule (Q4W) at a treatment dose of 1500 μg/kg.

In certain embodiments, the method comprises subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, then subcutaneously administering the BCMAxCD3 bispecific antibody on a weekly dosing schedule (QW) at a treatment dose of 1500 μg/kg for a period of time, then subcutaneously administering the BCMAxCD3 bispecific antibody on the bi-weekly dosing schedule (Q2W) at a treatment dose of 1500 μg/kg for at least 6 months, and then subcutaneously administering the BCMAxCD3 bispecific antibody on a monthly dosing schedule (Q4W) at a treatment dose of 1500 μg/kg.

In certain embodiments, the subject achieves a clinical response that is a PR, or a VGPR, or a CR or a sCR.

In certain embodiments, the subject achieves a clinical response that is a CR or a sCR.

In certain embodiments, the clinical response is maintained for at least 6 months.

In certain embodiments, the clinical response is maintained for at least 1 year.

In certain embodiments, the clinical response is maintained for at least 18 months.

In certain embodiments, the clinical response is maintained for at least 2 years.

In certain embodiments, fewer infections occur in the subject (e.g., compared to a subject that remains on QW dosing).

In certain embodiments, fewer grade ≥3 infections occur in the subject (e.g., compared to a subject that remains on QW dosing).

In certain embodiments, the method achieves a decrease in new infections over time, in a population of subjects with RRMM (e.g., compared to subjects that remain on a QW dosing schedule).

In certain embodiments, the method achieves a decrease in new grade ≥3 infections over time, in a population of subjects with RRMM (e.g., compared to subjects that remain on QW dosing schedule).

### Combination Regimens Comprising Daratumumab

According to certain embodiments, a BCMAxCD3 bispecific antibody as described herein (e.g., teclistamab) is administered as part of a combination regimen with daratumumab, wherein the BCMAxCD3 bispecific antibody is administered according to a dose schedule that comprises QW dosing, followed by Q2W dosing, followed by Q4W dosing. It is noted that Daratumumab SC is available as DARZALEX FASPRO®, and Teclistamab is available as TECVAYLI®. According to certain embodiments, the subject has received 1 to 3 prior line(s) of antimyeloma therapy including a proteasome inhibitor (PI) and lenalidomide. In certain embodiments, the subject has received only 1 line of prior line of antimyeloma therapy and is lenalidomide refractory.

According to certain embodiments, the daratumumab is administered subcutaneously. According to certain embodiments, each dose of daratumumab is subcutaneously administered in an amount of 1800 mg in 28-day treatment cycles, as follows:
Cycle 1 Day 1 and weekly (QW) thereafter (e.g., Days 1, 8, 15 and 22);
Cycle 2: weekly (QW) (e.g., Days 1, 8, 15 and 22);
Cycles 3-6: biweekly (Q2W) (e.g., Days 1 and 15);
Cycle 7 and subsequent cycles: monthly (Q4W) (e.g., Day 1).

According to certain embodiments, there are ≥5 days between doses of the daratumumab.

According to certain embodiments, the BCMAxCD3 bispecific antibody (e.g., teclistamab) is subcutaneously administered in 28-day treatment cycles, as follows:
Cycle 1: 0.06 mg/kg step-up dose on Day 2, 0.3 mg/kg step-up dose (e.g., on Day 4), 1.5 mg/kg step-up dose (e.g., on Day 8) and 1.5 mg/kg weekly (QW) treatment doses thereafter (e.g., Days 15 and 22);
Cycle 2: 1.5 mg/kg weekly (QW) (e.g., Days 1, 8, 15 and 22);
Cycles 3-6: 3 mg/kg biweekly (Q2W) (e.g., Days 1 and 15);
Cycle 7 and subsequent cycles: 3 mg/kg monthly (Q4W) (e.g., Day 1).

According to certain embodiments, the daratumumab is subcutaneously administered on Cycle 1 Day 1, and the first step-up dose of the BCMAxCD3 bispecific antibody is administered ≥20 hours after the daratumumab. According to certain embodiments, there are ≥2 days between step-up doses of the BCMAxCD3 bispecific antibody. According to certain embodiments, the first 1.5 mg/kg dose of teclistamab is administered ≥2 days after the second step-up dose. According to certain embodiments, there are ≥5 days between the 1.5 mg/kg treatment doses (or between a 1.5 mg/kg and the first 3 mg/kg dose) and ≥12 days between the 3 mg/kg treatment doses. According to certain embodiments, on the first day the daratumumab and the BCMAxCD3 bispecific antibody are administered on the same day (e.g., Cycle 1 Day 8), the BCMAxCD3 bispecific antibody is administered ≥3 hours after the daratumumab.

According to certain embodiments, a method of improving progression-free survival in a population of subjects with multiple myeloma that have received 1 to 3 prior line(s) of antimyeloma therapy, including a proteasome inhibitor (PI) and lenalidomide, comprises:
administering to the population of subjects a combination regimen comprising the teclistamab and daratumumab, wherein the teclistamab is subcutaneously administered in 28-day treatment cycles, as follows:
Cycle 1: 0.06 mg/kg step-up dose on Day 2, 0.3 mg/kg step-up dose (e.g., on Day 4), 1.5 mg/kg step-up dose (e.g., on Day 8) and 1.5 mg/kg weekly (QW) treatment doses thereafter (e.g., Days 15 and 22);
Cycle 2: 1.5 mg/kg weekly (QW) (e.g., Days 1, 8, 15 and 22);
Cycles 3-6: 3 mg/kg biweekly (Q2W) (e.g., Days 1 and 15);
Cycle 7 and subsequent cycles: 3 mg/kg monthly (Q4W) (e.g., Day 1); and
   wherein the daratumumab is subcutaneously administered in an amount of 1800 mg in the 28-day treatment cycles, as follows:

Cycle 1 Day 1 and weekly (QW) thereafter (e.g., Days 1, 8, 15 and 22);
Cycle 2: weekly (QW) (e.g., Days 1, 8, 15 and 22);
Cycles 3-6: biweekly (Q2W) (e.g., Days 1 and 15);
Cycle 7 and subsequent cycles: monthly (Q4W) (e.g., Day 1),
wherein the improvement in the median progression-free survival is relative to the median progression-free survival in a reference population of subjects, the reference population having been administered (i) a therapeutically effective regimen comprising Daratumumab SC, Pomalidomide, and Dexamethasone (DPd) (i.e., that does not include teclistamab) or (ii) a therapeutically effective regimen comprising Daratumumab SC, Bortezomib, and Dexamethasone (DVd) (i.e., that does not include teclistamab). According to certain embodiments, the improvement in the median progression-free survival is at least about 2 months, or at least about 4 months, or at least about 6 months, or at least about 8 months, or at least about 10 months, or at least about 12 months, or at least about 14 months, or at least about 16 months, or at least about 18 months, or at least about 20 months, or at least about 22 months, or at least about 24 months compared to the reference population.

### Prophylactic Administration of Toci

In MajesTEC-1, cytokine release syndrome (CRS) occurred in 72.1% of pts treated at the recommended phase 2 dose (RP2D) of weekly teclistamab 1.5 mg/kg (50.3% grade [gr] 1; 21.2% gr 2; 0.6% gr 3), and was successfully managed using Tocilizumab (“toci”) in 36.4% of pts (±other interventions) without affecting response to teclistamab. The inventors have discovered that prophylactic administration of toci prior to the first dose of a BCMAxCD3 bispecific antibody reduces CRS incidence and severity, which may facilitate outpatient initiation of therapy.

Embodiments of the present invention provide methods of treating multiple myeloma in a subject in need thereof by administering to the subject a therapeutically effective amount of tocilizumab prior to administering a first dose of the BCMAxCD3 bispecific antibody (e.g., teclistamab).

In particular embodiments, the patients are relapsed or refractory or intolerant to the last line of therapy (LOT); were exposed to a proteasome inhibitor, immunomodulatory drug, and anti-CD38 therapy; and had measurable disease.

In some embodiments, the subject has received three anti-cancer therapies prior to administration of the BCMAxCD3 bispecific antibody. In one embodiment, the three prior anti-cancer therapies are a proteasome inhibitor (PI), an immunomodulatory drug (IMiD), and an anti-CD38 antibody. In certain such embodiments, the proteasome inhibitor is bortezomib, carfilzomib or ixazomib, the immunomodulatory drug (IMiD) is lenalidomide, pomalidomide or thalidomide and the anti-CD38 antibody is daratumumab or isatuximab.

In certain embodiments, a single dose of toci before teclistamab treatment reduces the incidence of CRS relative to the overall MajesTEC-1 study population with no new safety signals and no evidence of impact on response to teclistamab. In certain embodiments, prophylactic toci reduces CRS risk in patients with disease profiles suitable for outpatient dosing, reducing the burden of hospitalization during teclistamab step-up dosing.

In certain embodiments, toci is administered prior to teclistamab as a single 8 mg/kg IV dose. In certain embodiments, teclistamab is administered subcutaneously according to a weight-based dosing schedule as shown in Table B, Table C, Table D, or Table E.

Methods of Restarting Therapy after a Dose Delay

Also provided herein are methods of restarting a subject's dosing regimen for a BCMAxCD3 bispecific antibody after the subject has initiated a planned dosing regimen for treatment of multiple myeloma (e.g., a dosing regimen as illustrated in Table F), and a delay has occurred between doses of the BCMAxCD3 bispecific antibody during the planned dosing regimen. According to certain embodiments, a planned dosing regimen comprises a step-up dosing schedule that includes a first step-up dose of 0.06 mg/kg, a second step-up dose of 0.3 mg/kg between 2 to 4 days (or between 2 to 7 days) after the first step-up dose, a first treatment dose of 1.5 mg/kg between 2 to 4 days (or between 2 to 7 days) after the second step-up dose; and subsequent treatment doses of 1.5 mg/kg one week after the first treatment dose and once weekly thereafter. According to certain embodiments, a planned dosing regimen comprises a step-up dosing schedule that includes a first step-up dose of 0.06 mg/kg, a second step-up dose of 0.3 mg/kg between 2 to 4 days (or between 2 to 7 days) after the first step-up dose, a first treatment dose of 1.5 mg/kg between 2 to 4 days (or between 2 to 7 days) after the second step-up dose; subsequent treatment doses of 1.5 mg/kg one week after the first treatment dose and once weekly thereafter; and if the subject has achieved and maintained a complete response or better for a minimum of 6 months, subsequent treatment doses of 1.5 mg/kg administered bi-weekly (every two weeks) (i.e., the dosing frequency may be decreased from 1.5 mg/kg weekly to 1.5 mg/kg every two weeks). According to certain embodiments, a planned dosing regimen comprises a step-up dosing schedule that includes a first step-up dose of 0.06 mg/kg, a second step-up dose of 0.3 mg/kg between 2 to 4 days (or between 2 to 7 days) after the first step-up dose, a first treatment dose of 1.5 mg/kg between 2 to 4 days (or between 2 to 7 days) after the second step-up dose; subsequent treatment doses of 1.5 mg/kg one week after the first treatment dose and once weekly thereafter; and if the subject has achieved and maintained any clinical response for a minimum of 6 months, subsequent treatment doses of 1.5 mg/kg administered bi-weekly (every two weeks) (i.e., the dosing frequency may be decreased to 1.5 mg/kg every two weeks).

Dosing regimens of a pharmaceutical product are developed based on extensive pre-clinical and clinical data. Once a safe and effective dosing regimen is established for a given therapy (also referred to herein as a planned dosing regimen), subjects must adhere to the regimen or otherwise face significant risks, such as increased side effects and/or diminished efficacy, etc. However, it is not uncommon for subjects to experience dose delays, especially a subject with multiple myeloma that faces day-to-day health and lifestyle challenges. When subjects experience a delay between doses of a BCMAxCD3 bispecific antibody that is longer than the interval between those doses in the established (planned) dosing regimen, there is a need for methods of restarting the therapy that are safe and effective. The inventors have developed novel methods of restarting therapy on a BCMAxCD3 bispecific antibody that are tailored to the length and timing of a particular dose delay, i.e., a method is selected from several options based on the last dose amount administered to the subject and the number of days since the last dose administered. The methods enable subjects to receive a dosing regimen that is safe and effective in treating multiple myeloma, despite experiencing a delay in dosing.

According to previous methods of restarting teclistamab, for example, if a patient experienced a dose delay after starting the 1.5 mg/kg treatment doses, and (1) if the dose delay was 28 days or less, the patient could continue teclistamab at their most recent treatment dose and schedule (1.5 mg/kg weekly or bi-weekly), or (2) if the dose delay was more than 28 days, the patient would have to restart the step-up dosing schedule at step-up dose 1 (0.06 mg/kg).

The inventors developed an optimized method of restarting teclistamab based, at least in part, on clinical data from patients in MajesTEC-1 that received more than one treatment dose before a prolonged dose delay. Incidence of cytokine release syndrome (CRS) and frequency of repeat step-up dosing (SUD) in patients with Tec dosing intervals >28 days were assessed. Of 61 subjects, with 128 intervals greater than 28 days, only 2 subjects had recurrence of CRS. Both subjects had intervals between >28-62 days, and both had 60 and 300 μg/kg step up doses. One subject had Grade 2 CRS at the start of Cycle 2 after 44 day delay, and one subject had Grade 1 CRS at the start of Cycle 6; both subjects had delays due infections. There were 100 intervals between >28-62 days, and in the majority of these, no repeat step up doses were given (78%); 78 subjects had intervals of >28-62 days with no repeat step up doses and no recurrent CRS upon restart. Based on the emerging safety data, the inventors developed a restart schedule that allows patients to resume their regimen at a more efficacious doses, e.g., the 1.5 mg/kg treatment dose or 0.3 mg/kg step-up dose 2, despite a dose delay that is longer than 28 days. According to certain embodiments, a patient that has started the 1.5 mg/kg treatment doses does not have to restart the regimen from the first 0.06 mg/kg step-up dose, unless the dose delay is more than 111 days (instead of more than 28 days). Compared to previous methods, the novel “restart” methods enable patients to safely resume their dosing regimens at more efficacious doses, even if they experience a dose delay that is longer than 28 days, e.g., if they experience a dose delay that is 62 days or less they can resume treatment dosing at 1.5 mg/kg, or if they experience a dose delay that is 63-111 days they can restart step-up dosing at 0.3 mg/kg.

According to certain embodiments, if a patient experiences a dose delay after starting the 1.5 mg/kg treatment doses, and (1) if the dose delay is 62 days or less (instead of 28 days or less), the patient can continue teclistamab at their most recent treatment dose and schedule (1.5 mg/kg weekly or bi-weekly), or (2) if the dose delay is 63 days to 111 days, the patient can restart the step-up dosing schedule at step-up dose 2 (0.3 mg/kg), or (3) if the dose delay is more than 111 days, the patient restarts the step-up dosing schedule at step-up dose 1 (0.06 mg/kg).

In accordance with certain embodiments of the present invention, a method of restarting teclistamab after a dose delay comprises continuing teclistamab at the same dose and schedule if the delay is up to 62 days, restarting teclistamab at step-up dose 2 (0.3 mg/kg) if the delay is between 63 and 111 days, and restarting teclistamab at step-up dose 1 (0.06 mg/kg) if the delay is more than 111 days.

According to certain embodiments, a method of restarting a subject's dosing regimen for a BCMAxCD3 bispecific antibody (e.g., teclistamab) after the subject has initiated a planned dosing regimen for treatment of multiple myeloma, and a delay has occurred between doses of the BCMAxCD3 bispecific antibody during the planned dosing regimen, wherein the planned dosing regimen comprises:
(1) a step-up dosing schedule that includes (a) a first step-up dose of 0.06 mg/kg, (b) a second step-up dose of 0.3 mg/kg between 2 to 7 days after the first step-up dose (e.g., between 2 to 4 days after the first step-up dose), and (c) a first treatment dose of 1.5 mg/kg between 2 to 7 days after the second step-up dose (e.g., between 2 to 4 days after the second step-up dose); and
(2) a weekly dosing schedule that includes subsequent treatment doses of 1.5 mg/kg one week after the first treatment dose and once weekly thereafter; and
(3) if the subject has achieved and maintained a clinical response for a minimum of 6 months (e.g., if the subject has achieved and maintained a complete response for a minimum of 6 months), a bi-weekly dosing schedule that includes subsequent treatment doses of 1.5 mg/kg once every two weeks,
then the method of restarting the subject's dosing regimen comprises:
(i) if the last dose administered to the subject was a treatment dose of 1.5 mg/kg, and the time from the last dose administered is 62 days or less, then continuing the planned dosing regimen at the last treatment dose and schedule (treatment doses of 1.5 mg/kg weekly or 1.5 mg/kg bi-weekly), or
(ii) if the last dose administered to the subject was a treatment dose of 1.5 mg/kg, and the time from the last dose administered is 63 days to 111 days, then restarting the step-up dosing schedule at the second step-up dose of 0.3 mg/kg and resuming the planned dosing regimen, or
(iii) if the last dose administered to the subject was a treatment dose of 1.5 mg/kg, and the time from the last dose administered is more than 111 days, then restarting the step-up dosing schedule at the first step-up dose of 0.06 mg/kg and resuming the planned dosing regimen.

According to certain embodiments, if a dose of the BCMAxCD3 bispecific antibody (e.g., teclistamab) is delayed, the patient restarts therapy based on Table 7 and resumes the weekly or bi-weekly (every 2 weeks) dosing schedule accordingly.

TABLE 7
Recommendations for Restarting Teclistamab after Dose Delay

| | Duration of delay | |
|---|---|---|
| Last dose | from the last dose | |
| administered | administered | Action |
| Step-up | More than 7 days | Restart teclistamab step-up dosing |
| dose 1 | | schedule at step-up |
| | | dose 1 (0.06 mg/kg). |
| Step-up | 8 days to 28 days | Repeat step-up dose 2 (0.3 mg/kg) |
| dose 2 | | and continue teclistamab step-up |
| | | dosing schedule. |
| | More than 28 days^{a} | Restart teclistamab step-up dosing |
| | | schedule at step-up dose 1 |
| | | (0.06 mg/kg). |
| Any | 62 days or less | Continue teclistamab at last |
| treatment | | treatment dose and schedule (1.5 |
| dose | | mg/kg once weekly or 1.5 mg/kg |
| | | every two weeks). |
| | 63 days to 111 days | Restart teclistamab step-up dosing |
| | | schedule at step-up dose 2 (0.3 |
| | | mg/kg). |
| | More than 111 days^{b} | Restart teclistamab step-up dosing |
| | | schedule at step-up dose 1 |
| | | (0.06 mg/kg). |

^{a}Consider benefit-risk of restarting teclistamab in patients who require a dose delay of more than 28 days due to an adverse reaction.
^{b}Consider benefit-risk of restarting teclistamab in patients who require a dose delay of more than 111 days due to an adverse reaction

### Exemplary Embodiments

Provided below are enumerated embodiments of the present invention. These embodiments are illustrative only and do not limit the scope of the present disclosure or of the claims attached hereto.
1A. A method of treating multiple myeloma in a subject in need thereof, comprising administering to the subject a therapeutically effective amount of a BCMAxCD3 bispecific antibody (e.g., teclistamab) on a bi-weekly dosing schedule (Q2W), wherein the subject has relapsed or refractory multiple myeloma and received at least three prior lines of therapy, including a proteasome inhibitor, an immunomodulatory agent and an anti-CD38 monoclonal antibody.
2A. The method of embodiment 1A, wherein the BCMAxCD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.
3A. The method of embodiment 2A, wherein the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.
4A. The method of any of embodiments 1A-3A, wherein the BCMAxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.
5A. The method of any of embodiments 1A-4A, wherein the BCMAxCD3 bispecific antibody is teclistamab.
6A. The method of any of embodiments 1A-5A comprising administering a treatment dose of the BCMAxCD3 bispecific antibody once every two weeks.
7A. The method of embodiment 6A comprising administering one or more step-up doses of the BCMAxCD3 bispecific antibody prior to administering a treatment dose of the BCMAxCD3 bispecific antibody.
8A. The method of any of embodiments 1A-7A comprising administering treatment doses of the BCMAxCD3 bispecific antibody once per week and subsequently administering treatment doses of the BCMAxCD3 bispecific antibody once every two weeks.
9A. The method of any of embodiments 1A-7A comprising administering the BCMAxCD3 bispecific antibody according to the following dosing schedule:

| | | |
|---|---|---|
| Dosing | | |
| Schedule | Day | Dose |

| | | | |
|---|---|---|---|
| Step-up | Day 1 | Step-up | 0.06 mg/kg |
| Dosing | | dose 1 | |
| Schedule | Day 4 | Step-up | 0.3 mg/kg |
| | | dose 2 | |
| | Day 7 | First | 1.5 mg/kg |
| | | treatment | |
| | | dose | |
| Weekly | One week after | Subsequent | 1.5 mg/kg |
| Dosing | first treatment dose | treatment | once every |
| Schedule | and once every two | doses | two weeks |
| | weeks thereafter | | |

10A. The method of any of embodiments 1A-7A comprising administering the BCMAxCD3 bispecific antibody according to the following dosing schedule:

| | | |
|---|---|---|
| Dosing | | |
| Schedule | Day | Dose |

All Patients

| | | | |
|---|---|---|---|
| Step-up Dosing | Day 1 | Step-up | 0.06 mg/kg |
| Schedule | | dose 1 | |
| | Day 4^{a} | Step-up | 0.3 mg/kg |
| | | dose 2 | |
| | Day 7^{b} | First | 1.5 mg/kg |
| | | treatment | |
| | | dose | |
| Weekly Dosing | One week after | Subsequent | 1.5 mg/kg |
| Schedule | first treatment | treatment | once |
| | dose and | doses | weekly |
| | weekly | | |
| | thereafter | | |

Patients who have a response for a minimum of 6 months

| Biweekly (every | Consider reducing the dosing frequency |
|---|---|
| two weeks) | to 1.5 mg/kg every two weeks |
| dosing schedule | |

11A. The method of any of embodiments 1A-10A, wherein the BCMAxCD3 bispecific antibody is administered subcutaneously.
12A. The method of any of embodiments 1A-5A comprising subcutaneously administering the BCMAxCD3 bispecific antibody biweekly (Q2W) at a treatment dose of 1500 μg/kg (e.g., Days 1 and 15 of each 28-day cycle).
13A. The method of any of embodiments 1A-5A comprising subcutaneously administering the BCMAxCD3 bispecific antibody monthly (Q4W) at a treatment dose of 1500 μg/kg (e.g., Day 1 of each 28-day cycle).
14A. The method of any of embodiments 1A-5A comprising subcutaneously administering the BCMAxCD3 bispecific antibody weekly (QW) at a treatment dose of 1500 μg/kg (e.g., Days 1, 8, 15, and 22 of each 28-day cycle) for a period of time, and then subcutaneously administering the BCMAxCD3 bispecific antibody biweekly (Q2W) at a treatment dose of 1500 μg/kg (e.g., Days 1 and 15 of each 28-day cycle).
15A. The method of any of embodiments 1A-5A comprising subcutaneously administering the BCMAxCD3 bispecific antibody weekly (QW) at a treatment dose of 1500 μg/kg (e.g., Days 1, 8, 15, and 22 of each 28-day cycle) for at least 6 months, and then subcutaneously administering the BCMAxCD3 bispecific antibody biweekly (Q2W) at a treatment dose of 1500 μg/kg (e.g., Days 1 and 15 of each 28-day cycle).
16A. The method of any of embodiments 1A-5A comprising subcutaneously administering the BCMAxCD3 bispecific antibody weekly (QW) at a treatment dose of 1500 μg/kg (e.g., Days 1, 8, 15, and 22 of each 28-day cycle) for at least 6 months, and then subcutaneously administering the BCMAxCD3 bispecific antibody biweekly (Q2W) at a treatment dose of 1500 μg/kg (e.g., Days 1 and 15 of each 28-day cycle) after the subject has achieved a PR, a VGPR, a CR or a sCR.
17A. The method of any of embodiments 1A-5A comprising subcutaneously administering the BCMAxCD3 bispecific antibody weekly (QW) at a treatment dose of 1500 μg/kg (e.g., Days 1, 8, 15, and 22 of each 28-day cycle) for at least 4 cycles, and then subcutaneously administering the BCMAxCD3 bispecific antibody biweekly (Q2W) at a treatment dose of 1500 μg/kg (e.g., Days 1 and 15 of each 28-day cycle) after the subject has achieved a PR, a VGPR, a CR or a sCR.
18A. The method of any of embodiments 1A-5A comprising subcutaneously administering the BCMAxCD3 bispecific antibody weekly (QW) at a treatment dose of 1500 μg/kg (e.g., Days 1, 8, 15, and 22 of each 28-day cycle) for at least 6 months, and then subcutaneously administering the BCMAxCD3 bispecific antibody biweekly (Q2W) at a treatment dose of 1500 μg/kg (e.g., Days 1 and 15 of each 28-day cycle) after the subject has achieved a CR or a sCR.
19A. The method of any of embodiments 1A-5A comprising subcutaneously administering the BCMAxCD3 bispecific antibody weekly (QW) at a treatment dose of 1500 μg/kg (e.g., Days 1, 8, 15, and 22 of each 28-day cycle) for at least 4 cycles, and then subcutaneously administering the BCMAxCD3 bispecific antibody biweekly (Q2W) at a treatment dose of 1500 μg/kg (e.g., Days 1 and 15 of each 28-day cycle) after the subject has achieved a CR or a sCR.
20A. The method of any of embodiments 1A-19A, wherein the subject achieves a clinical response that is a PR, or a VGPR, or a CR or a sCR following initiation of the Q2W administration of the treatment doses.
21A. The method of any of embodiments 1A-19A, wherein the subject achieves a clinical response that is a PR, or a VGPR, or a CR or a sCR following initiation of the Q4W administration of the treatment doses.
22A. The method of any of embodiments 20A-21A, wherein the clinical response is maintained for at least 6 months.
23A. The method of any of embodiments 20A-21A, wherein the clinical response is maintained for at least 1 year.
24A. The method of any of embodiments 20A-21A, wherein the clinical response is maintained for at least 18 months.
25A. The method of any of embodiments 20A-21A, wherein the clinical response is maintained for at least 2 years.
26A. The method of any of embodiments 1A-25A, wherein fewer infections occur in the subject (e.g., compared to a subject that remains on QW dosing).
27A. The method of any of embodiments 1A-25A, wherein fewer grade ≥3 infections occur in the subject (e.g., compared to a subject that remains on QW dosing).

Additional enumerated embodiments of the present invention are provided below. These embodiments are illustrative only and do not limit the scope of the present disclosure or of the claims attached hereto.
1B. A method of treating multiple myeloma in a subject in need thereof, comprising administering to the subject a therapeutically effective amount of a BCMAxCD3 bispecific antibody (e.g., teclistamab) on a bi-weekly dosing schedule (Q2W) and/or a monthly dosing schedule (Q4W), wherein the subject has been diagnosed with multiple myeloma.
2B. The method of embodiment 1B, wherein the subject has relapsed or refractory multiple myeloma and received at least three prior lines of therapy, including a proteasome inhibitor, an immunomodulatory agent and an anti-CD38 monoclonal antibody.
3B. The method of embodiment 1B or 2B, wherein the BCMAxCD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.
4B. The method of embodiment 1B or 2B, wherein the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.
5B. The method of any of embodiments 1B-4B, wherein the BCMAxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.
6B. The method of any of embodiments 1B-4B, wherein the BCMAxCD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.
7B. The method of any of embodiments 1B-5B, wherein the BCMAxCD3 bispecific antibody is teclistamab.
8B. The method of any of embodiments 1B-6B comprising administering a treatment dose of the BCMAxCD3 bispecific antibody once every two weeks.
9B. The method of embodiment 8B comprising administering one or more step-up doses of the BCMAxCD3 bispecific antibody prior to administering a treatment dose of the BCMAxCD3 bispecific antibody.
10B. The method of any of embodiments 1B-9B comprising administering treatment doses of the BCMAxCD3 bispecific antibody once per week and subsequently administering treatment doses of the BCMAxCD3 bispecific antibody once every two weeks.
11B. The method of any of embodiments 1B-9B comprising administering the BCMAxCD3 bispecific antibody according to the following dosing schedule:

| | | |
|---|---|---|
| Dosing | | |
| Schedule | Day | Dose |

| | | | |
|---|---|---|---|
| Step-up | Day 1 | Step-up | 0.06 mg/kg |
| Dosing | | dose 1 | |
| Schedule | Day 4 | Step-up | 0.3 mg/kg |
| | | dose 2 | |
| | Day 7 | First | 1.5 mg/kg |
| | | treatment | |
| | | dose | |
| Weekly | One week after | Subsequent | 1.5 mg/kg |
| Dosing | first treatment dose | treatment | once every |
| Schedule | and once every two | doses | two weeks |
| | weeks thereafter | | |

12B. The method of any of embodiments 1B-9B comprising administering the BCMAxCD3 bispecific antibody according to the following dosing schedule:

| | | |
|---|---|---|
| Dosing | | |
| Schedule | Day | Dose |

All Patients

| | | | |
|---|---|---|---|
| Step-up Dosing | Day 1 | Step-up | 0.06 mg/kg |
| Schedule | | dose 1 | |
| | Day 4^{a} | Step-up | 0.3 mg/kg |
| | | dose 2 | |
| | Day 7^{b} | First | 1.5 mg/kg |
| | | treatment | |
| | | dose | |
| Weekly Dosing | One week after | Subsequent | 1.5 mg/kg |
| Schedule | first treatment | treatment | once |
| | dose and | doses | weekly |
| | weekly | | |
| | thereafter | | |

Patients who have a response for a minimum of 6 months

| Biweekly (every | Consider reducing the dosing frequency to |
|---|---|
| two weeks) | 1.5 mg/kg every two weeks |
| dosing schedule | |

13B. The method of any of embodiments 1B-12B, wherein the BCMAxCD3 bispecific antibody is administered subcutaneously.
14B. The method of any of embodiments 1B-7B comprising subcutaneously administering the BCMAxCD3 bispecific antibody biweekly (Q2W) at a treatment dose of 1500 μg/kg (e.g., Days 1 and 15 of each 28-day cycle).
15B. The method of any of embodiments 1B-7B comprising subcutaneously administering the BCMAxCD3 bispecific antibody monthly (Q4W) at a treatment dose of 1500 μg/kg (e.g., Day 1 of each 28-day cycle).
16B. The method of any of embodiments 1B-7B comprising subcutaneously administering the BCMAxCD3 bispecific antibody weekly (QW) at a treatment dose of 1500 μg/kg (e.g., Days 1, 8, 15, and 22 of each 28-day cycle) for a period of time, and then subcutaneously administering the BCMAxCD3 bispecific antibody biweekly (Q2W) at a treatment dose of 1500 μg/kg (e.g., Days 1 and 15 of each 28-day cycle).
17B. The method of any of embodiments 1B-7B comprising subcutaneously administering the BCMAxCD3 bispecific antibody weekly (QW) at a treatment dose of 1500 μg/kg (e.g., Days 1, 8, 15, and 22 of each 28-day cycle) for at least 6 months, and then subcutaneously administering the BCMAxCD3 bispecific antibody biweekly (Q2W) at a treatment dose of 1500 μg/kg (e.g., Days 1 and 15 of each 28-day cycle).
18B. The method of any of embodiments 1B-7B comprising subcutaneously administering the BCMAxCD3 bispecific antibody weekly (QW) at a treatment dose of 1500 μg/kg (e.g., Days 1, 8, 15, and 22 of each 28-day cycle) for at least 6 months, and then subcutaneously administering the BCMAxCD3 bispecific antibody biweekly (Q2W) at a treatment dose of 1500 μg/kg (e.g., Days 1 and 15 of each 28-day cycle) after the subject has achieved a PR, a VGPR, a CR or a sCR.
19B. The method of any of embodiments 1B-7B comprising subcutaneously administering the BCMAxCD3 bispecific antibody weekly (QW) at a treatment dose of 1500 μg/kg (e.g., Days 1, 8, 15, and 22 of each 28-day cycle) for at least 4 cycles, and then subcutaneously administering the BCMAxCD3 bispecific antibody biweekly (Q2W) at a treatment dose of 1500 μg/kg (e.g., Days 1 and 15 of each 28-day cycle) after the subject has achieved a PR, a VGPR, a CR or a sCR.
20B. The method of any of embodiments 1B-7B comprising subcutaneously administering the BCMAxCD3 bispecific antibody weekly (QW) at a treatment dose of 1500 μg/kg (e.g., Days 1, 8, 15, and 22 of each 28-day cycle) for at least 6 months, and then subcutaneously administering the BCMAxCD3 bispecific antibody biweekly (Q2W) at a treatment dose of 1500 μg/kg (e.g., Days 1 and 15 of each 28-day cycle) after the subject has achieved a CR or a sCR.
21B. The method of any of embodiments 1B-7B comprising subcutaneously administering the BCMAxCD3 bispecific antibody weekly (QW) at a treatment dose of 1500 μg/kg (e.g., Days 1, 8, 15, and 22 of each 28-day cycle) for at least 4 cycles, and then subcutaneously administering the BCMAxCD3 bispecific antibody biweekly (Q2W) at a treatment dose of 1500 μg/kg (e.g., Days 1 and 15 of each 28-day cycle) after the subject has achieved a CR or a sCR.
22B. The method of any of embodiments 1B-21B, wherein the subject achieves a clinical response that is a PR, or a VGPR, or a CR or a sCR following initiation of the Q2W administration of the treatment doses.
23B. The method of any of embodiments 1B-21B, wherein the subject achieves a clinical response that is a PR, or a VGPR, or a CR or a sCR following initiation of the Q4W administration of the treatment doses.
24B. The method of any of embodiments 22B-23B, wherein the clinical response is maintained for at least 6 months.
25B. The method of any of embodiments 22B-23B, wherein the clinical response is maintained for at least 1 year.
26B. The method of any of embodiments 22B-23B, wherein the clinical response is maintained for at least 18 months.
27B. The method of any of embodiments 22B-23B, wherein the clinical response is maintained for at least 2 years.
28B. The method of any of embodiments 1B-27B, wherein fewer infections occur in the subject (e.g., compared to a subject that remains on QW dosing).
29B. The method of any of embodiments 1B-27B, wherein fewer grade ≥3 infections occur in the subject (e.g., compared to a subject that remains on QW dosing).

Additional enumerated embodiments of the present invention are provided below. These embodiments are illustrative only and do not limit the scope of the present disclosure or of the claims attached hereto.
1C. A method of treating multiple myeloma in a subject in need thereof, comprising administering to the subject a therapeutically effective amount of tocilizumab and subsequently administering a therapeutically effective amount of a BCMAxCD3 bispecific antibody (e.g., teclistamab), wherein the subject has relapsed or refractory multiple myeloma and received at least three prior lines of therapy, including a proteasome inhibitor, an immunomodulatory agent and an anti-CD38 monoclonal antibody.
2C. The method of embodiment 1C, wherein the BCMAxCD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.
3C. The method of embodiment 2C, wherein the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.
4C. The method of any of embodiments 1C-3C, wherein the BCMAxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.
5C. The method of any of embodiments 1C-4C, wherein the BCMAxCD3 bispecific antibody is teclistamab.
6C. The method of any of embodiments 1C-5C, wherein the method reduces the risk of CRS in the subject compared to a subject that has not received toci prior to receiving the BCMAxCD3 bispecific antibody.
7C. The method of any of embodiments 1C-6C, wherein the tocilizumab is administered about 4 hours or less before a first teclistamab step-up dose (e.g., from about 2 hours to about 4 hours before a first teclistamab step-up dose).
8C. The method of any of embodiments 1C-7C, wherein the tocilizumab is administered as a single 8 mg/kg IV dose.
9C. The method of any of embodiments 1C-8C, wherein the method achieves a CRS rate in a population of subjects that is lower than a reference CRS rate in a reference population of subjects, the reference population of subjects having not received tocilizumab prior to receiving the BCMAxCD3 bispecific antibody.

Additional enumerated embodiments of the present invention are provided below. These embodiments are illustrative only and do not limit the scope of the present disclosure or of the claims attached hereto.
1D. A method of restarting a subject's dosing regimen for a BCMAxCD3 bispecific antibody (e.g., teclistamab) after the subject has initiated a planned dosing regimen for treatment of multiple myeloma, and a delay has occurred between doses of the BCMAxCD3 bispecific antibody during the planned dosing regimen, wherein the planned dosing regimen comprises:
   (1) a step-up dosing schedule that includes (a) a first step-up dose of 0.06 mg/kg, (b) a second step-up dose of 0.3 mg/kg between 2 to 7 days after the first step-up dose (e.g., between 2 to 4 days after the first step-up dose), and (c) a first treatment dose of 1.5 mg/kg between 2 to 7 days after the second step-up dose (e.g., between 2 to 4 days after the second step-up dose); and
   (2) a weekly dosing schedule that includes subsequent treatment doses of 1.5 mg/kg one week after the first treatment dose and once weekly thereafter; and
   (3) if the subject has achieved and maintained a clinical response for a minimum of 6 months (e.g., if the subject has achieved and maintained a complete response for a minimum of 6 months), a bi-weekly dosing schedule that includes subsequent treatment doses of 1.5 mg/kg once every two weeks,
   wherein the method of restarting the subject's dosing regimen comprises:
   (i) if the last dose administered to the subject was a treatment dose of 1.5 mg/kg, and the time from the last dose administered is 62 days or less, then continuing the planned dosing regimen at the last treatment dose and schedule (treatment doses of 1.5 mg/kg weekly or 1.5 mg/kg bi-weekly), or
   (ii) if the last dose administered to the subject was a treatment dose of 1.5 mg/kg, and the time from the last dose administered is 63 days to 111 days, then restarting the step-up dosing schedule at the second step-up dose of 0.3 mg/kg and resuming the planned dosing regimen, or
   (iii) if the last dose administered to the subject was a treatment dose of 1.5 mg/kg, and the time from the last dose administered is more than 111 days, then restarting the step-up dosing schedule at the first step-up dose of 0.06 mg/kg and resuming the planned dosing regimen.
2D. The method of embodiment 1D, wherein the method of restarting the subject's dosing regimen comprises:
   (i) if the last dose administered to the subject was a treatment dose of 1.5 mg/kg, and the time from the last dose administered is 62 days or less, then continuing the planned dosing regimen at the last treatment dose and schedule (treatment doses of 1.5 mg/kg weekly or 1.5 mg/kg bi-weekly), or
   (ii) if the last dose administered to the subject was a treatment dose of 1.5 mg/kg, and the time from the last dose administered is 63 days to 111 days, then restarting the step-up dosing schedule at the second step-up dose of 0.3 mg/kg and resuming the planned dosing regimen, or
   (iii) if the last dose administered to the subject was a treatment dose of 1.5 mg/kg, and the time from the last dose administered is more than 111 days, then restarting the step-up dosing schedule at the first step-up dose of 0.06 mg/kg and resuming the planned dosing regimen, or
   (iv) if the last dose administered to the subject was the first step-up dose of 0.06 mg/kg, and the time from the last dose administered is more than 7 days, then restarting the step-up dosing schedule at the first step-up dose of 0.06 mg/kg and resuming the planned dosing regimen, or
   (v) if the last dose administered to the subject was the second step-up dose of 0.3 mg/kg, and the time from the last dose administered is 8 to 28 days, then repeating the second step-up dose of 0.3 mg/kg and continuing the step-up dosing schedule (thereby resuming the planned dosing regimen), or
   (vi) if the last dose administered to the subject was the second step-up dose of 0.3 mg/kg, and the time from the last dose administered is more than 28 days, then restarting the step-up dosing schedule at the first step-up dose of 0.06 mg/kg and resuming the planned dosing regimen.
3D. The method of embodiment 1D or 2D, wherein the planned dosing regimen comprises the bi-weekly dosing schedule that includes treatment doses of 1.5 mg/kg once every two weeks, if the subject has achieved and maintained a complete response for a minimum of 6 months.
4D. The method of any of embodiments 1D-3D, wherein the step-up dosing schedule includes (a) the first step-up dose of 0.06 mg/kg on Day 1, (b) the second step-up dose of 0.3 mg/kg on Day 4, and (c) the first treatment dose of 1.5 mg/kg on Day 7.
5D. The method of any of embodiments 1D-3D, wherein each of the second step-up dose and the first treatment dose is administered between 2 to 4 days after the previous dose, or is optionally administered up to 7 days after the previous dose to allow for resolution of adverse reactions.
6D. The method of any of embodiments 1D-5D further comprising administering pretreatment medications to the subject prior to restarting the planned dosing regimen, wherein the pretreatment medications comprise 16 mg or equivalent of oral or intravenous dexamethasone, 50 mg or equivalent of oral or intravenous diphenhydramine, and 650 mg to 1000 mg, or equivalent, oral or intravenous acetaminophen.
7D. The method of any of embodiments 1D-6D, wherein the BCMAxCD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.
8D. The method of any of embodiments 1D-7D, wherein the BCMAxCD3 bispecific antibody is an IgG1, an IgG2, an IgG3 or an IgG4 isotype.
9D. The method of any of embodiments 1D-8D, wherein the BCMAxCD3 bispecific antibody is an IgG4 isotype.
10D. The method of any of embodiments 1D-9D, wherein the BCMAxCD3 bispecific antibody comprises one or more substitutions in its Fc region.
11D. The method of any of embodiments 1D-10D, wherein the BCMAxCD3 bispecific antibody is an IgG4 isotype and comprises Proline/Alanine/Alanine substitutions at amino acid positions 228/234/235, respectively, in its Fc region (according to EU index numbering).
12D. The method of any of embodiments 1D-11D, wherein the BCMAxCD3 bispecific antibody is an IgG4 isotype and comprises F405L and R409K substitutions in its Fc region (according to EU index numbering).
13D. The method of any of embodiments 1D-12D, wherein the Fc region of the BCMA-binding arm comprises Proline/Alanine/Alanine substitutions at amino acid positions 228/234/235, respectively (according to EU index numbering).
14D. The method of any of embodiments 1D-13D, wherein the Fc region of the CD3-binding arm comprises Proline/Alanine/Alanine substitutions at amino acid positions 228/234/235, respectively, in addition to F405L and R409K substitutions (according to EU index numbering).
15D. The method of any of embodiments 1D-14D, wherein the BCMAxCD3 bispecific antibody comprises a BCMA binding domain and a CD3 binding domain, wherein the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.
16D. The method of any of embodiments 1D-15D, wherein the BCMAxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.
17D. The method of any of embodiments 1D-15D, wherein the BCMAxCD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO:22 and a second light chain (LC2) having at least 95% identity to the amino acid sequence of SEQ ID NO: 23.
18D. The method of any of embodiments 1D-15D, wherein the BCMAxCD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.
19D. The method of any of embodiments 1D-15D, wherein the BCMAxCD3 bispecific antibody comprises a first heavy chain (HC1) having at least 99% identity to the amino acid sequence of SEQ ID NO: 12, a first light chain (LC1) having at least 99% identity to the amino acid sequence of SEQ ID NO: 13, a second heavy chain (HC2) having at least 99% identity to the amino acid sequence of SEQ ID NO: 22 and a second light chain (LC2) having at least 99% identity to the amino acid sequence of SEQ ID NO: 23.
20D. The method of any of embodiments 1D-15D, wherein the BCMAxCD3 bispecific antibody is teclistamab.
21D. The method of any of embodiments 1D-20D, wherein the subject has relapsed or refractory multiple myeloma.
22D. The method of any of embodiments 1D-21D, wherein the subject has relapsed and refractory multiple myeloma and has received at least 3 prior therapies, including an immunomodulatory agent, a proteasome inhibitor, and an anti-CD38 antibody and has demonstrated disease progression on the last therapy.
23D. The method of any of embodiments 1D-21D, wherein the subject has relapsed or refractory multiple myeloma and has received at least four prior lines of therapy, including a proteasome inhibitor, an immunomodulatory agent and an anti-CD38 monoclonal antibody.

Additional enumerated embodiments of the present invention are provided below. These embodiments are illustrative only and do not limit the scope of the present disclosure or of the claims attached hereto.

1. A method of treating multiple myeloma in a subject in need thereof, comprising administering to the subject treatment doses of a BCMAxCD3 bispecific antibody on a bi-weekly dosing schedule (Q2W).

2. The method of embodiment 1, wherein the BCMAxCD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19.

3. The method of embodiment 1 or 2, wherein the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.

4. The method of any of embodiments 1-3, wherein the BCMAxCD3 bispecific antibody is an IgG1, an IgG2, an IgG3 or an IgG4 isotype.

5. The method of any of embodiments 1-4, wherein the BCMAxCD3 bispecific antibody is an IgG4 isotype.

6. The method of any of embodiments 1-5, wherein the BCMAxCD3 bispecific antibody comprises one or more substitutions in its Fc region.

7. The method of any of embodiments 1-6, wherein the BCMAxCD3 bispecific antibody is an IgG4 isotype and comprises S228P, F234A and L235A substitutions in its Fc region (according to EU numbering).

8. The method of any of embodiments 1-7, wherein the BCMAxCD3 bispecific antibody is an IgG4 isotype and comprises S228P, F234A, L235A F405L and R409K substitutions in its Fc region (according to EU numbering).

9. The method of any of embodiments 1-8, wherein the Fc region of the BCMA-binding arm comprises S228P, F234A and L235A substitutions in its Fc region (according to EU numbering).

10. The method of any of embodiments 1-9, wherein the Fc region of the CD3-binding arm comprises S228P, F234A, L235A, F405L, and R409K substitutions in its Fc region (according to EU numbering).

11. The method of any of embodiments 1-10, wherein the BCMAxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.

12. The method of any of embodiments 1-10, wherein the BCMAxCD3 bispecific antibody comprises a first heavy chain (HC1) having at least 90% identity to the amino acid sequence of SEQ ID NO: 12, a first light chain (LC1) having at least 90% identity to the amino acid sequence of SEQ ID NO: 13, a second heavy chain (HC2) having at least 90% identity to the amino acid sequence of SEQ ID NO: 22 and a second light chain (LC2) having at least 90% identity to the amino acid sequence of SEQ ID NO: 23.

13. The method of any of embodiments 1-10, wherein the BCMAxCD3 bispecific antibody comprises a first heavy chain (HC1) having at least 95% identity to the amino acid sequence of SEQ ID NO: 12, a first light chain (LC1) having at least 95% identity to the amino acid sequence of SEQ ID NO: 13, a second heavy chain (HC2) having at least 95% identity to the amino acid sequence of SEQ ID NO: 22 and a second light chain (LC2) having at least 95% identity to the amino acid sequence of SEQ ID NO: 23.

14. The method of any of embodiments 1-10, wherein the BCMAxCD3 bispecific antibody comprises a first heavy chain (HC1) having at least 98% identity to the amino acid sequence of SEQ ID NO: 12, a first light chain (LC1) having at least 98% identity to the amino acid sequence of SEQ ID NO: 13, a second heavy chain (HC2) having at least 98% identity to the amino acid sequence of SEQ ID NO: 22 and a second light chain (LC2) having at least 98% identity to the amino acid sequence of SEQ ID NO: 23.

15. The method of any of embodiments 1-10, wherein the BCMAxCD3 bispecific antibody is teclistamab.

16. The method of any of embodiments 1-15, wherein the subject has relapsed or refractory multiple myeloma myeloma (e.g., the subject has had been treated with from 2 to 14 prior lines of therapy).

17. The method of any of embodiments 1-16, wherein the subject has received at least three prior lines of therapy.

18. The method of any of embodiments 1-16, wherein the subject has received at least four prior lines of therapy.

19. The method of any of embodiments 1-16, wherein the subject has received at least five prior lines of therapy (penta-drug exposed).

20. The method of any of embodiments 1-16, wherein the subject has received at least three prior lines of therapy, including a proteasome inhibitor, an immunomodulatory agent and an anti-CD38 monoclonal antibody.

21. The method of any of embodiments 1-16, wherein the subject has received at least four prior lines of therapy, including a proteasome inhibitor, an immunomodulatory agent and an anti-CD38 monoclonal antibody.

22. The method of any of embodiments 1-21 comprising subcutaneously administering treatment doses of the BCMAxCD3 bispecific antibody on the bi-weekly dosing schedule (Q2W).

23. The method of any of embodiments 1-22 comprising subcutaneously administering to the subject one or more step-up doses of the BCMAxCD3 bispecific antibody prior to administering the first treatment dose of the BCMAxCD3 bispecific antibody.

24. The method of any of embodiments 1-23 comprising subcutaneously administering the BCMAxCD3 bispecific antibody on the bi-weekly dosing schedule (Q2W) at a treatment dose of from about 1500 μg/kg to about 3000 μg/kg.

25. The method of any of embodiments 1-23 comprising subcutaneously administering the BCMAxCD3 bispecific antibody on the bi-weekly dosing schedule (Q2W) at a treatment dose of about 1500 μg/kg.

26. The method of any of embodiments 1-23 comprising subcutaneously administering the BCMAxCD3 bispecific antibody on the bi-weekly dosing schedule (Q2W) at a treatment dose of about 3000 μg/kg.

27. The method of any of embodiments 1-26 comprising subcutaneously administering to the subject at least one treatment dose of the BCMAxCD3 bispecific antibody on a weekly dosing schedule (QW) and subsequently administering to the subject treatment doses of the BCMAxCD3 bispecific antibody on the bi-weekly dosing schedule (Q2W).

28. The method of any of embodiments 1-27 comprising subcutaneously administering treatment doses of the BCMAxCD3 bispecific antibody on a weekly dosing schedule (QW) for at least one 28-day cycle of treatment, and subsequently administering treatment doses of the BCMAxCD3 bispecific antibody on the bi-weekly dosing schedule (Q2W).

29. The method of any of embodiments 1-27 comprising subcutaneously administering treatment doses of the BCMAxCD3 bispecific antibody on a weekly dosing schedule (QW) for at least two 28-day cycles of treatment, and subsequently administering treatment doses of the BCMAxCD3 bispecific antibody on the bi-weekly dosing schedule (Q2W).

30. The method of any of embodiments 1-27 comprising subcutaneously administering treatment doses of the BCMAxCD3 bispecific antibody on a weekly dosing schedule (QW) for at least three 28-day cycles of treatment, and subsequently administering treatment doses of the BCMAxCD3 bispecific antibody on the bi-weekly dosing schedule (Q2W).

31. The method of any of embodiments 1-27 comprising subcutaneously administering treatment doses of the BCMAxCD3 bispecific antibody on a weekly dosing schedule (QW) for at least four 28-day cycles of treatment, and subsequently administering treatment doses of the BCMAxCD3 bispecific antibody on the bi-weekly dosing schedule (Q2W).

32. The method of any of embodiments 1-27 comprising subcutaneously administering treatment doses of the BCMAxCD3 bispecific antibody on a weekly dosing schedule (QW) for at least five 28-day cycles of treatment, and subsequently administering treatment doses of the BCMAxCD3 bispecific antibody on the bi-weekly dosing schedule (Q2W).

33. The method of any of embodiments 1-27 comprising subcutaneously administering treatment doses of the BCMAxCD3 bispecific antibody on a weekly dosing schedule (QW) for at least six 28-day cycles of treatment, and subsequently administering treatment doses of the BCMAxCD3 bispecific antibody on the bi-weekly dosing schedule (Q2W).

34. The method of any of embodiments 1-27 comprising subcutaneously administering treatment doses of the BCMAxCD3 bispecific antibody on a weekly dosing schedule (QW) for at least six months, and subsequently administering treatment doses of the BCMAxCD3 bispecific antibody on the bi-weekly dosing schedule (Q2W).

35. The method of any of embodiments 1-34 comprising subcutaneously administering to the subject treatment doses of the BCMAxCD3 bispecific antibody on a weekly dosing schedule (QW) until the subject achieves a partial response, a very good partial response, a complete response or a stringent complete response, as determined by IMWG response criteria, and subsequently administering to the subject treatment doses of the BCMAxCD3 bispecific antibody on the bi-weekly dosing schedule (Q2W).

36. The method of any of embodiments 1-34 comprising subcutaneously administering to the subject treatment doses of the BCMAxCD3 bispecific antibody on a weekly dosing schedule (QW) until the subject achieves a very good partial response, a complete response or a stringent complete response, as determined by IMWG response criteria, and subsequently administering to the subject treatment doses of the BCMAxCD3 bispecific antibody on the bi-weekly dosing schedule (Q2W).

37. The method of any of embodiments 1-34 comprising subcutaneously administering to the subject treatment doses of the BCMAxCD3 bispecific antibody on a weekly dosing schedule (QW) until the subject achieves a complete response or a stringent complete response, as determined by IMWG response criteria, and subsequently administering to the subject treatment doses of the BCMAxCD3 bispecific antibody on the bi-weekly dosing schedule (Q2W).

38. The method of any of embodiments 1-27 comprising subcutaneously administering to the subject treatment doses of the BCMAxCD3 bispecific antibody on a weekly dosing schedule (QW) until the subject achieves a partial response, a very good partial response, a complete response or a stringent complete response after four or more cycles of treatment, as determined by IMWG response criteria, and subsequently administering to the subject treatment doses of the BCMAxCD3 bispecific antibody on the bi-weekly dosing schedule (Q2W).

39. The method of any of embodiments 1-27 comprising subcutaneously administering to the subject treatment doses of the BCMAxCD3 bispecific antibody on a weekly dosing schedule (QW) until (i) the subject achieves a partial response, a very good partial response, a complete response or a stringent complete response for six or more months, as determined by IMWG response criteria, or (ii) the subject achieves a complete response or a stringent complete response for six or more months, as determined by IMWG response criteria, and
subsequently administering to the subject treatment doses of the BCMAxCD3 bispecific antibody on the bi-weekly dosing schedule (Q2W).

40. The method of any of embodiments 27-39 wherein each treatment dose amount of the BCMAxCD3 bispecific antibody administered on the weekly dosing schedule (QW) is the same treatment dose amount of the BCMAxCD3 bispecific antibody administered on the bi-weekly dosing schedule (Q2W).

41. The method of any of embodiments 27-40 comprising subcutaneously administering the BCMAxCD3 bispecific antibody on the weekly dosing schedule (QW) at a treatment dose of from about 1500 μg/kg to about 3000 μg/kg.

42. The method of any of embodiments 27-40 comprising subcutaneously administering the BCMAxCD3 bispecific antibody on the weekly dosing schedule (QW) at a treatment dose of about 1500 μg/kg.

43. The method of any of embodiments 27-40 comprising subcutaneously administering the BCMAxCD3 bispecific antibody on the weekly dosing schedule (QW) at a treatment dose of about 3000 μg/kg.

44. The method of any of embodiments 27-40 comprising subcutaneously administering the BCMAxCD3 bispecific antibody on the weekly dosing schedule (QW) at a treatment dose of about 1500 μg/kg, and then subcutaneously administering the BCMAxCD3 bispecific antibody on the bi-weekly dosing schedule (Q2W) at a treatment dose of about 1500 μg/kg.

45. The method of any of embodiments 27-40 comprising subcutaneously administering the BCMAxCD3 bispecific antibody on the weekly dosing schedule (QW) at a treatment dose of about 1500 μg/kg, and then subcutaneously administering the BCMAxCD3 bispecific antibody on the bi-weekly dosing schedule (Q2W) at a treatment dose of about 3000 μg/kg.

46. The method of any of embodiments 1-45 comprising subcutaneously administering the BCMAxCD3 bispecific antibody on the bi-weekly dosing schedule (Q2W), and then subcutaneously administering the BCMAxCD3 bispecific antibody on a monthly dosing schedule (Q4W).

47. The method of any of embodiments 1-46 comprising subcutaneously administering the BCMAxCD3 bispecific antibody on a weekly dosing schedule (QW), and then subcutaneously administering the BCMAxCD3 bispecific antibody on the bi-weekly dosing schedule (Q2W), and then subcutaneously administering the BCMAxCD3 bispecific antibody on a monthly dosing schedule (Q4W).

48. The method of any of embodiments 1-47 comprising subcutaneously administering the BCMAxCD3 bispecific antibody on the bi-weekly dosing schedule (Q2W), and if the subject achieves a partial response, a very good partial response, a complete response or a stringent complete response, then subcutaneously administering the BCMAxCD3 bispecific antibody on a monthly dosing schedule (Q4W).

49. The method of any of embodiments 1-47 comprising subcutaneously administering the BCMAxCD3 bispecific antibody on the bi-weekly dosing schedule (Q2W), and if the subject achieves a complete response or a stringent complete response, then subcutaneously administering the BCMAxCD3 bispecific antibody on a monthly dosing schedule (Q4W).

50. The method of any of embodiments 1-47 comprising subcutaneously administering the BCMAxCD3 bispecific antibody on the bi-weekly dosing schedule (Q2W), and if the subject achieves a complete response or a stringent complete response by cycle 12 of treatment or later, then subcutaneously administering the BCMAxCD3 bispecific antibody on a monthly dosing schedule (Q4W).

51. The method of any of embodiments 1-47 comprising subcutaneously administering the BCMAxCD3 bispecific antibody on the bi-weekly dosing schedule (Q2W) for at least six months, and then subcutaneously administering the BCMAxCD3 bispecific antibody on a monthly dosing schedule (Q4W).

52. The method of any of embodiments 1-51 comprising subcutaneously administering 1, 2 or 3 step-up doses of the BCMAxCD3 bispecific antibody prior to subcutaneously administering the first treatment dose.

53. The method of any of embodiments 1-52 comprising subcutaneously administering step-up doses of 60 μg/kg and 300 μg/kg and 1500 μg/kg of the BCMAxCD3 bispecific antibody prior to subcutaneously administering the first treatment dose.

54. The method of any of embodiments 1-52 comprising subcutaneously administering 1 or 2 step-up doses of the BCMAxCD3 bispecific antibody prior to subcutaneously administering the first treatment dose.

55. The method of any of embodiments 1-52 comprising subcutaneously administering 2 step-up doses of the BCMAxCD3 bispecific antibody prior to subcutaneously administering the first treatment dose.

56. The method of any of embodiments 1-52 comprising subcutaneously administering step-up doses of 60 μg/kg and 300 μg/kg of the BCMAxCD3 bispecific antibody prior to subcutaneously administering the first treatment dose.

57. The method of any of embodiments 1-56 comprising subcutaneously administering step-up doses of the BCMAxCD3 bispecific antibody 2-4 days apart from each other, prior to subcutaneously administering the first treatment dose.

58. The method of any of embodiments 1-57 comprising subcutaneously administering one or more step-up doses of the BCMAxCD3 bispecific antibody prior to subcutaneously administering the first treatment dose, wherein the first treatment dose is administered 2-4 days after the last step-up dose.

59. The method of any of embodiments 1-58 comprising subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering the first treatment dose of the BCMAxCD3 bispecific antibody.

60. The method of any of embodiments 1-59 comprising subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg for a period of time, and then subcutaneously administering the BCMAxCD3 bispecific antibody on the bi-weekly dosing schedule (Q2W) at a treatment dose of 1500 μg/kg.

61. The method of any of embodiments 1-59 comprising subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg for six months or more, and then subcutaneously administering the BCMAxCD3 bispecific antibody on the bi-weekly dosing schedule (Q2W) at a treatment dose of 1500 μg/kg.

62. The method of any of embodiments 1-59 comprising subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg, and after the subject has a partial response, a very good partial response, a complete response or a stringent complete response for six months or more (according to IMWG criteria), then subcutaneously administering the BCMAxCD3 bispecific antibody on the bi-weekly dosing schedule (Q2W) at a treatment dose of 1500 μg/kg.

63. The method of any of embodiments 1-59 comprising subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg, and after the subject has a complete response or a stringent complete response for six months or more (according to IMWG criteria), then subcutaneously administering the BCMAxCD3 bispecific antibody on the bi-weekly dosing schedule (Q2W) at a treatment dose of 1500 μg/kg.

64. The method of any of embodiments 1-59 comprising subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, and then subcutaneously administering the BCMAxCD3 bispecific antibody on a weekly dosing schedule (QW) at a treatment dose of 1500 μg/kg, and after the subject has a partial response, a very good partial response, a complete response or a stringent complete response after four 28-day treatment cycles or more (according to IMWG criteria), then subcutaneously administering the BCMAxCD3 bispecific antibody on the bi-weekly dosing schedule (Q2W) at a treatment dose of 1500 μg/kg.

65. The method of any of embodiments 1-64 comprising subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, then subcutaneously administering the BCMAxCD3 bispecific antibody on a weekly dosing schedule (QW) at a treatment dose of 1500 μg/kg for a period of time, then subcutaneously administering the BCMAxCD3 bispecific antibody on the bi-weekly dosing schedule (Q2W) at a treatment dose of 1500 μg/kg for a period of time, and then subcutaneously administering the BCMAxCD3 bispecific antibody on a monthly dosing schedule (Q4W) at a treatment dose of 1500 μg/kg.

66. The method of any of embodiments 1-64 comprising subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, then subcutaneously administering the BCMAxCD3 bispecific antibody on a weekly dosing schedule (QW) at a treatment dose of 1500 μg/kg for a period of time, then subcutaneously administering the BCMAxCD3 bispecific antibody on the bi-weekly dosing schedule (Q2W) at a treatment dose of 1500 μg/kg for a period of time, and if the subject achieves at least CR (i.e., complete response or stringent complete response) by cycle 12 or later, then subcutaneously administering the BCMAxCD3 bispecific antibody on a monthly dosing schedule (Q4W) at a treatment dose of 1500 μg/kg.

67. The method of any of embodiments 1-64 comprising subcutaneously administering a first step-up dose of 60 μg/kg of the BCMAxCD3 bispecific antibody, then 2-4 days later subcutaneously administering a second step-up dose of 300 μg/kg of the BCMAxCD3 bispecific antibody, then 2-4 days later subcutaneously administering a first treatment dose of 1500 μg/kg of the BCMAxCD3 bispecific antibody, then subcutaneously administering the BCMAxCD3 bispecific antibody on a weekly dosing schedule (QW) at a treatment dose of 1500 μg/kg for a period of time, then subcutaneously administering the BCMAxCD3 bispecific antibody on the bi-weekly dosing schedule (Q2W) at a treatment dose of 1500 μg/kg for at least 6 months, and then subcutaneously administering the BCMAxCD3 bispecific antibody on a monthly dosing schedule (Q4W) at a treatment dose of 1500 μg/kg.

68. The method of any of embodiments 1-67, wherein the subject achieves a clinical response that is a PR, or a VGPR, or a CR or a sCR.

69. The method of any of embodiments 1-67, wherein the subject achieves a clinical response that is a CR or a sCR.

70. The method of embodiment 68 or 69, wherein the clinical response is maintained for at least 6 months.

71. The method of embodiment 68 or 69, wherein the clinical response is maintained for at least 1 year.

72. The method of embodiment 68 or 69, wherein the clinical response is maintained for at least 18 months.

73. The method of embodiment 68 or 69, wherein the clinical response is maintained for at least 2 years.

74. The method of any of embodiments 1-73, wherein fewer infections occur in the subject (e.g., compared to a subject that remains on QW dosing).

75. The method of any of embodiments 1-74, wherein fewer grade ≥3 infections occur in the subject (e.g., compared to a subject that remains on QW dosing).

76. The method of any of embodiments 1-73, wherein the method achieves a decrease in new infections over time, in a population of subjects with RRMM (e.g., compared to subjects that remain on a QW dosing schedule).

77. The method of any of embodiments 1-73, wherein the method achieves a decrease in new grade ≥3 infections over time, in a population of subjects with RRMM RRMM (e.g., compared to subjects that remain on QW dosing schedule).

78. The method of any of embodiments 1-77, further comprising administering to the subject a therapeutically effective amount of tocilizumab prior to administering a first dose of the BCMAxCD3 bispecific antibody (e.g., teclistamab).

79. The method of embodiment 78, wherein the method reduces the risk of CRS in the subject compared to a subject that has not received tocilizumab prior to receiving the BCMAxCD3 bispecific antibody.

80. The method of embodiment 78 or 79, wherein the tocilizumab is administered about 4 hours or less before a first teclistamab step-up dose (e.g., from about 2 hours to about 4 hours before a first teclistamab step-up dose).

81. The method of any of embodiments 78-80, wherein the tocilizumab is administered as a single 8 mg/kg IV dose.

82. The method of any of embodiments 78-81, wherein the method achieves a CRS rate in a population of subjects that is lower than a reference CRS rate in a reference population of subjects, the reference population of subjects having not received tocilizumab prior to receiving the BCMAxCD3 bispecific antibody.

83. The method of any of any of embodiments 1-82, comprising administering a combination regimen comprising the BCMAxCD3 bispecific antibody and daratumumab.

84. The method of embodiment 83, wherein the subject has received 1 to 3 prior line(s) of antimyeloma therapy including a proteasome inhibitor (PI) and lenalidomide.

85. The method of embodiment 84, wherein the subject has received only 1 line of prior line of antimyeloma therapy and is lenalidomide refractory.

86. The method of any of any of embodiments 83-85, wherein the daratumumab is administered subcutaneously.

87. The method of any of any of embodiments 82-86, wherein the daratumumab is subcutaneously administered in an amount of 1800 mg in 28-day treatment cycles, as follows:
Cycle 1 Day 1 and weekly (QW) thereafter (e.g., Days 1, 8, 15 and 22);
Cycle 2: weekly (QW) (e.g., Days 1, 8, 15 and 22);
Cycles 3-6: biweekly (Q2W) (e.g., Days 1 and 15);
Cycle 7 and subsequent cycles: monthly (Q4W) (e.g., Day 1).

88. The method of embodiment 87, wherein there are ≥5 days between doses of the daratumumab.

89. The method of any of embodiments 1-15, wherein the subject has received 1 to 3 prior line(s) of antimyeloma therapy including a proteasome inhibitor (PI) and lenalidomide, the method comprising:
administering to the subject a combination regimen comprising the BCMAxCD3 bispecific antibody (e.g., teclistamab) and daratumumab, wherein the BCMAxCD3 bispecific antibody is subcutaneously administered in 28-day treatment cycles, as follows:
Cycle 1: 0.06 mg/kg step-up dose on Day 2, 0.3 mg/kg step-up dose (e.g., on Day 4), 1.5 mg/kg step-up dose (e.g., on Day 8) and 1.5 mg/kg weekly (QW) treatment doses thereafter (e.g., Days 15 and 22);
Cycle 2: 1.5 mg/kg weekly (QW) (e.g., Days 1, 8, 15 and 22);
Cycles 3-6: 3 mg/kg biweekly (Q2W) (e.g., Days 1 and 15);
Cycle 7 and subsequent cycles: 3 mg/kg monthly (Q4W) (e.g., Day 1).

90. The method of embodiment 89, wherein the daratumumab is subcutaneously administered on Cycle 1 Day 1, and the first step-up dose of the BCMAxCD3 bispecific antibody is administered ≥20 hours after the daratumumab.

91. The method of embodiment 89 or 90, where there are ≥2 days between step-up doses of the BCMAxCD3 bispecific antibody.

92. The method of any of embodiments 89-91, wherein the first 1.5 mg/kg dose of teclistamab is administered ≥2 days after the second step-up dose.

93. The method of any of embodiments 89-92, wherein there are ≥5 days between the 1.5 mg/kg treatment doses (or between a 1.5 mg/kg and the first 3 mg/kg dose) and ≥12 days between the 3 mg/kg treatment doses.

94. The method of any of embodiments 89-93, wherein the daratumumab is subcutaneously administered in an amount of 1800 mg in the 28-day treatment cycles, as follows:
Cycle 1 Day 1 and weekly (QW) thereafter (e.g., Days 1, 8, 15 and 22);
Cycle 2: weekly (QW) (e.g., Days 1, 8, 15 and 22);
Cycles 3-6: biweekly (Q2W) (e.g., Days 1 and 15);
Cycle 7 and subsequent cycles: monthly (Q4W) (e.g., Day 1).

95. The method of any of embodiments 89-94, wherein on the first day the daratumumab and the BCMAxCD3 bispecific antibody are administered on the same day (e.g., Cycle 1 Day 8), the BCMAxCD3 bispecific antibody is administered ≥3 hours after the daratumumab.

96. The method of any of embodiments 89-95, wherein the BCMAxCD3 bispecific antibody is teclistamab.

97. A method of improving progression-free survival in a population of subjects with multiple myeloma that have received 1 to 3 prior line(s) of antimyeloma therapy including a proteasome inhibitor (PI) and lenalidomide, the method comprising: administering to the population of subjects a combination regimen comprising the teclistamab and daratumumab, wherein the teclistamab is subcutaneously administered in 28-day treatment cycles, as follows:
Cycle 1: 0.06 mg/kg step-up dose on Day 2, 0.3 mg/kg step-up dose (e.g., on Day 4), 1.5 mg/kg step-up dose (e.g., on Day 8) and 1.5 mg/kg weekly (QW) treatment doses thereafter (e.g., Days 15 and 22);
Cycle 2: 1.5 mg/kg weekly (QW) (e.g., Days 1, 8, 15 and 22);
Cycles 3-6: 3 mg/kg biweekly (Q2W) (e.g., Days 1 and 15);
Cycle 7 and subsequent cycles: 3 mg/kg monthly (Q4W) (e.g., Day 1); and wherein the daratumumab is subcutaneously administered in an amount of 1800 mg in the 28-day treatment cycles, as follows:
Cycle 1 Day 1 and weekly (QW) thereafter (e.g., Days 1, 8, 15 and 22);
Cycle 2: weekly (QW) (e.g., Days 1, 8, 15 and 22);
Cycles 3-6: biweekly (Q2W) (e.g., Days 1 and 15);
Cycle 7 and subsequent cycles: monthly (Q4W) (e.g., Day 1),
wherein the improvement in the median progression-free survival is relative to the median progression-free survival in a reference population of subjects, the reference population having been administered (i) a therapeutically effective regimen comprising Daratumumab SC, Pomalidomide, and Dexamethasone (DPd) or (ii) a therapeutically effective regimen comprising Daratumumab SC, Bortezomib, and Dexamethasone (DVd).

98. The method of embodiment 97, wherein the improvement in the median progression-free survival is at least about 2 months, or at least about 4 months, or at least about 6 months, or at least about 8 months, or at least about 10 months, or at least about 12 months, or at least about 14 months, or at least about 16 months, or at least about 18 months, or at least about 20 months, or at least about 22 months, or at least about 24 months.

99. The method of embodiment 97 or 98, wherein the first step-up dose of the teclistamab is administered ≥20 hours after the daratumumab.

100. The method of any of embodiments 97-99, wherein on the first day the daratumumab and the teclistamab are administered on the same day (e.g., Cycle 1 Day 8), the teclistamab is administered ≥3 hours after the daratumumab.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

### EXAMPLES

The following examples are provided to further describe some of the embodiments disclosed herein. The examples are intended to illustrate, not to limit, the disclosed embodiments.

### Example 1: Durability of Responses with Biweekly Dosing of Teclistamab in Patients with Relapsed/Refractory Multiple Myeloma (RRMM) Achieving a Clinical Response in the MajesTEC-1 Study

### Antibodies

Anti-BCMA/anti-CD3 antibody teclistamab (also called Tec) (e.g., described in WO2017031104A1, the content of which is incorporated herein by reference in its entirety) was made by Janssen Pharmaceuticals. Teclistamab comprises a BCMA binding arm BCMB69 and a CD3 binding arm CD3B219, the amino acid sequences of which are shown in Table 5 and Table 6, respectively.

TABLE 5
Sequences of BCMA binding arm of Teclistamab

| | | | SEQ ID |
|---|---|---|---|
| | Region | Sequence | NO: |
| BCMB69 | HCDR1 | SGSYFWG | 4 |
| | HCDR2 | SIYYSGITYYNPSLKS | 5 |
| | HCDR3 | HDGAVAGLFDY | 6 |
| | LCDR1 | GGNNIGSKSVH | 7 |
| | LCDR2 | DDSDRPS | 8 |
| | LCDR3 | QVWDSSSDHVV | 9 |
| | VH | QLQLQESGPGLVKPSETLSLTCTVSGGSISSGSY | 10 |
| | | FWGWIRQPPGKGLEWIGSIYYSGITYYNPSLKS | |
| | | RVTISVDTSKNQFSLKLSSVTAADTAVYYCAR | |
| | | HDGAVAGLFDYWGQGTLVTVSS | |
| | VL | SYVLTQPPSVSVAPGQTARITCGGNNIGSKSVH | 11 |
| | | WYQQPPGQAPVVVVYDDSDRPSGIPERFSGSN | |
| | | SGNTATLTISRVEAGDEAVYYCQVWDSSSDHV | |
| | | VFGGGTKLTVL | |
| | HC | QLQLQESGPGLVKPSETLSLTCTVSGGSISSGSY | 12 |
| | | FWGWIRQPPGKGLEWIGSIYYSGITYYNPSLKS | |
| | | RVTISVDTSKNQFSLKLSSVTAADTAVYYCAR | |
| | | HDGAVAGLFDYWGQGTLVTVSSASTKGPSVFP | |
| | | LAPCSRSTSESTAALGCLVKDYFPEPVTVSWNS | |
| | | GALTSGVHTFPAVLQSSGLYSLSSVVTVPSSSL | |
| | | GTKTYTCNVDHKPSNTKVDKRVESKYGPPCPP | |
| | | CPAPEAAGGPSVFLFPPKPKDTLMISRTPEVTCV | |
| | | VVDVSQEDPEVQFNWYVDGVEVHNAKTKPRE | |
| | | EQFNSTYRVVSVLTVLHQDWLNGKEYKCKVS | |
| | | NKGLPSSIEKTISKAKGQPREPQVYTLPPSQEEM | |
| | | TKNQVSLTCLVKGFYPSDIAVEWESNGQPENN | |
| | | YKTTPPVLDSDGSFFLYSRLTVDKSRWQEGNV | |
| | | FSCSVMHEALHNHYTQKSLSLSLGK | |
| | LC | SYVLTQPPSVSVAPGQTARITCGGNNIGSKSVH | 13 |
| | | WYQQPPGQAPVVVVYDDSDRPSGIPERFSGSN | |
| | | SGNTATLTISRVEAGDEAVYYCQVWDSSSDHV | |
| | | VFGGGTKLTVLGQPKAAPSVTLFPPSSEELQAN | |
| | | KATLVCLISDFYPGAVTVAWKGDSSPVKAGVE | |
| | | TTTPSKQSNNKYAASSYLSLTPEQWKSHRSYSC | |
| | | QVTHEGSTVEKTVAPTECS | |

TABLE 6
Sequences of CD3 binding arm of Teclistamab

| | | | SEQ |
|---|---|---|---|
| | Region | Sequence | ID NO: |
| CD3B219 | HCDR1 | TYAMN | 14 |
| | HCDR2 | RIRSKYNNYATYYAASVKG | 15 |
| | HCDR3 | HGNFGNSYVSWFAY | 16 |
| | LCDR1 | RSSTGAVTTSNYAN | 17 |
| | LCDR2 | GTNKRAP | 18 |
| | LCDR3 | ALWYSNLWV | 19 |
| | VH | EVQLVESGGGLVQPGGSLRLSCAASGFTFNT | 20 |
| | | YAMNWVRQAPGKGLEWVARIRSKYNNYAT | |
| | | YYAASVKGRFTISRDDSKNSLYLQMNSLKTE | |
| | | DTAVYYCARHGNFGNSYVSWFAYWGQGTL | |
| | | VTVSS | |
| | VL | QTVVTQEPSLTVSPGGTVTLTCRSSTGAVTT | 21 |
| | | SNYANWVQQKPGQAPRGLIGGTNKRAPGTP | |
| | | ARFSGSLLGGKAALTLSGVQPEDEAEYYCAL | |
| | | WYSNLWVFGGGTKLTVLGQP | |
| | HC | EVQLVESGGGLVQPGGSLRLSCAASGFTFNT | 22 |
| | | YAMNWVRQAPGKGLEWVARIRSKYNNYAT | |
| | | YYAASVKGRFTISRDDSKNSLYLQMNSLKTE | |
| | | DTAVYYCARHGNFGNSYVSWFAYWGQGTL | |
| | | VTVSSASTKGPSVFPLAPCSRSTSESTAALGC | |
| | | LVKDYFPEPVTVSWNSGALTSGVHTFPAVL | |
| | | QSSGLYSLSSVVTVPSSSLGTKTYTCNVDHK | |
| | | PSNTKVDKRVESKYGPPCPPCPAPEAAGGPS | |
| | | VFLFPPKPKDTLMISRTPEVTCVVVDVSQED | |
| | | PEVQFNWYVDGVEVHNAKTKPREEQFNSTY | |
| | | RVVSVLTVLHQDWLNGKEYKCKVSNKGLPS | |
| | | SIEKTISKAKGQPREPQVYTLPPSQEEMTKNQ | |
| | | VSLTCLVKGFYPSDIAVEWESNGQPENNYKT | |
| | | TPPVLDSDGSFLLYSKLTVDKSRWQEGNVFS | |
| | | CSVMHEALHNHYTQKSLSLSLGK | |
| | LC | QTVVTQEPSLTVSPGGTVTLTCRSSTGAVTT | 23 |
| | | SNYANWVQQKPGQAPRGLIGGTNKRAPGTP | |
| | | ARFSGSLLGGKAALTLSGVQPEDEAEYYCAL | |
| | | WYSNLWVFGGGTKLTVLGQPKAAPSVTLFP | |
| | | PSSEELQANKATLVCLISDFYPGAVTVAWKA | |
| | | DSSPVKAGVETTTPSKQSNNKYAASSYLSLT | |
| | | PEQWKSHRSYSCQVTHEGSTVEKTVAPTECS | |

### Aims:

To evaluate the ability of patients to maintain their responses after transitioning from weekly (QW) to every other week (Q2W) dosing schedules in the pivotal phase 1/2 MajesTEC-1 trial (NCT03145181/NCT04557098). 5

### Methods

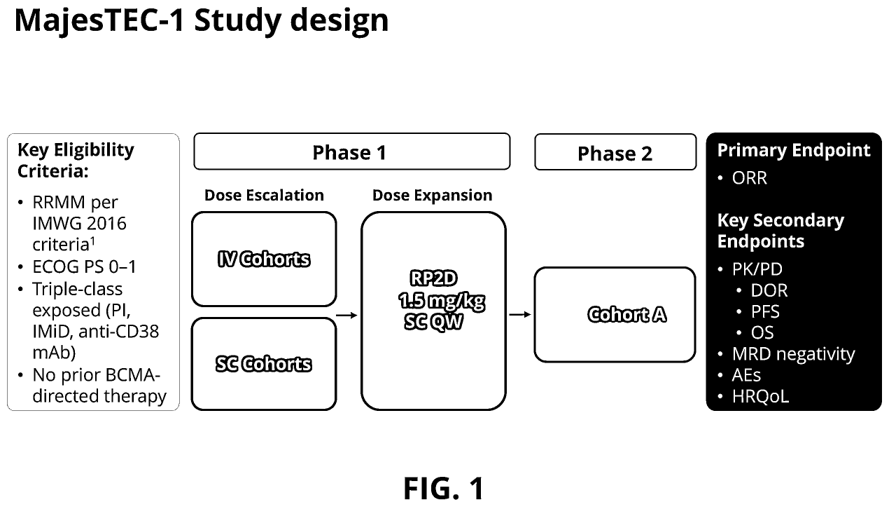

Eligible patients had RRMM and received ≥3 prior lines of therapy including a proteasome inhibitor, immunomodulatory drug, and anti-CD38 antibody. Prior BCMA-targeted therapy was not allowed in this cohort. All patients provided informed consent. Patients in MajesTEC-1 who achieved and maintained a response could switch to a less frequent dosing schedule, offering added convenience and flexibility to patients, physicians, and caregivers; and the durability of responses in patients who switched from QW to Q2W or Q4W dosing schedules in MajesTEC-1 were assessed. Specifically, patients received the recommended phase 2 dose (RP2D) of 1.5 mg/kg teclistamab QW, with the option to switch to Q2W dosing if patients achieved a confirmed partial response or better after ≥4 cycles of treatment (phase 1) or a confirmed complete response (CR) or better for ≥6 months (phase 2). Response was assessed per IMWG 2016 criteria. An illustration of the study design is provided in FIG. 1.

### Dosing Regimens:

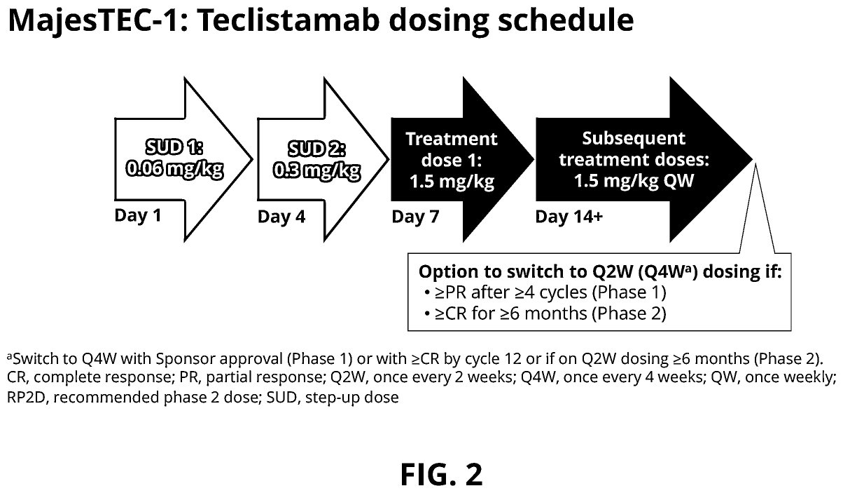

Eligible patients received teclistamab at the RP2D (1.5 mg/kg teclistamab QW), with the option for responders to switch to Q2W or Q4W dosing. An illustration of dose escalations is provided in FIG. 2.

### Dose Schedules:

Weight-based RP2D: Teclistamab SC (subcutaneous) at 60 and 300 μg/kg (priming doses) followed by a 1500 μg/kg weekly treatment dose.

Priming (Step-Up) Dose Schedule: The priming schedule includes 2 priming doses of 60 and 300 μg/kg. Each priming dose is separated by 2 to 4 days and to be completed 2 to 4 days prior to the first treatment dose. If there are no delays in treatment, the first treatment dose should be administered 4 to 8 days after the first priming dose and 2 to 4 days after the second priming dose.

Treatment Dose Schedule: full dose (1500 μg/kg) on each dosing day:
Weekly (QW) dosing: Days 1, 8, 15, and 22 of a 28-day cycle.
Biweekly (Q2W) dosing: Cycle Days 1 and 15 of a 28-day cycle.
Monthly (Q4W) dosing: Cycle Day 1 of a 28-day cycle.

For any subjects receiving the weekly dosing, a change from the weekly dosing to the biweekly dosing schedule was permitted. Patients had the option to switch from QW to Q2W dosing if:
achieved at least PR (i.e., partial response, very good partial response, complete response or stringent complete response) after at least 4 cycles (Phase 1), or
achieved at least CR (i.e., complete response or stringent complete response) for at least 6 months (Phase 2).

Patients had the option to switch to Q4W dosing if:
Sponsor approval was received (Phase 1), or
achieved at least CR (i.e., complete response or stringent complete response) by cycle 12 or later (Phase 2), or
if on Q2W dosing for at least 6 months (Phase 2).

Primary Outcome Measures: Overall Response Rate (ORR) [Time Frame: Up to 2.9 years]. ORR is defined as the proportion of participants who have a partial response (PR) or better according to the International Myeloma Working Group (IMWG) criteria.

### Eligibility Criteria:

### Inclusion Criteria

Documented diagnosis of multiple myeloma according to IMWG diagnostic criteria
Eastern Cooperative Oncology Group (ECOG) Performance Status score of 0 or 1
Measurable disease: Cohort A, Cohort C and Cohort D: Multiple myeloma must be measurable by central laboratory assessment
A female participant of childbearing potential must have a negative pregnancy test at screening
Willing and able to adhere to the prohibitions and restrictions specified in this protocol
Cohorts A and D: received at least 3 prior MM treatment lines of therapy. Prior therapy must include an IMiD, PI, and anti-CD38 monoclonal antibody; Cohort C: received >=3 prior lines of therapy that included a PI, an IMiD, an anti-CD38 monoclonal antibody, and an anti-B cell maturation antigen (BCMA) treatment (with CART-T cells or an antibody drug conjugate (ADC)

### Exclusion Criteria

Plasma cell leukemia, Waldenström's macroglobulinemia, POEMS syndrome, or primary amyloid light-chain amyloidosis
The following medical conditions: Pulmonary compromise requiring supplemental oxygen use to maintain adequate oxygenation, human immunodeficiency virus (HIV) infection, hepatitis B or C infection, stroke or seizure less than or equal to (<=) 6 m, autoimmune disease, uncontrolled systemic infection, cardiac conditions (Myocardial Infarction <=6 m, stage III-IV congestive heart failure, etc)
Received any therapy that is targeted to BCMA, with the exception of Cohort C in Part 3.

Prior antitumor therapy, within 21 days (PI or radiotherapy within 14 days, IMiDs within 7 days, Gene modified adoptive cell therapy within 3 months) prior to first dose of study drug
Toxicities from previous anticancer therapies that have not resolved to baseline or to <=grade 1 (except for alopecia or peripheral neuropathy)
Received a cumulative dose of corticosteroids equivalent to >=140 mg of prednisone within the 14-day period before the first dose of study drug (does not include pretreatment medication)
Known active central nervous system (CNS) involvement or exhibits clinical signs of meningeal involvement of multiple myeloma (MM)
Myelodysplastic syndrome or active malignancies other than relapsed/refractory multiple myeloma with exceptions are: 1) Non-muscle invasive bladder cancer treated within the last 24 months that is considered completely cured 2) Skin cancer (non-melanoma or melanoma) treated within the last 24 months that is considered completely cured. 3) Noninvasive cervical cancer treated within the last 24 months that is considered completely cured. 4) Localized prostate cancer (N0M0) 5) Breast cancer: Adequately treated lobular carcinoma in situ or ductal carcinoma in situ, or history of localized breast cancer and receiving antihormonal agents and considered to have a very low risk of recurrence. 6) Malignancy that is considered cured with minimal risk of recurrence
Prior allogenic stem cell transplant <=6 months
Prior autologous stem cell transplant <=12 weeks
Live, attenuated vaccine within 4 weeks prior to the first dose of teclistamab

### Results as of Data Cut-Off Dec. 9, 2022:

As of Dec. 9, 2022, 165 patients in the pivotal cohort had received teclistamab at the RP2D. Of 104 responders, 60 patients switched to Q2W dosing; 50 met the protocol-defined criteria for switching, and 10 switched who did not meet the criteria (4 due to adverse events [AEs]; 6 due to other reasons). Patients who switched had a median age of 64 years, 58% were male, 25% had high-risk cytogenetics, 7% had extramedullary plasmacytomas, and 3% had International Staging System stage III disease at baseline. Patients received a median of 4 prior lines of therapy, and 75% were triple-class refractory. At the time of switch, 49 (82%) patients achieved ≥CR, and 11 (18%) had a very good partial response. Median time to switch from QW to Q2W dosing was 11.1 months (range, 3-20). At median 11.1-month (range, 2-24) follow-up since switching, the median duration of response from the date of switch was 20.5 months (range, 1-23), with 40/60 patients still in response and ongoing treatment. Of the remaining patients, 13/60 have progressed (median time from switch to progression not estimable), 2 discontinued due to AEs, 1 discontinued for other reason, and 4 died. Additional results will be presented.

### SUMMARY/CONCLUSION

Overall, patients from the MajesTEC-1 study who transitioned from QW to less frequent Q2W dosing of teclistamab had sustained remission, with a median duration of response of 20.5 months from the date of switch.

### Results as of Data Cut-Off Jan. 4, 2023:

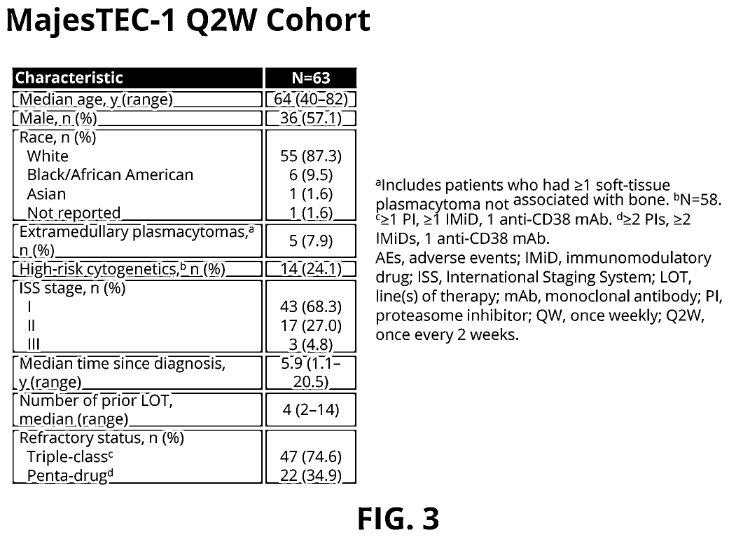

Characteristics of MajesTEC-1 patients that switched to less frequent dosing are summarized in FIG. 3. As of Jan. 4, 2023 (median follow-up: 22.8 months), 165 patients had received teclistamab in the pivotal cohort. Of the 104 responders, 63 switched to Q2W dosing (22 from Phase 1; 41 from Phase 2) and 9 responders switched to Q4W. Fifty-four (54) patients met the protocol-defined criteria for switching; nine (9) switched who did not meet the criteria, due to AEs (n=3) or other reasons (n-6). Median time to switch from first dose on QW schedule to first dose on Q2W schedule: 11.3 months (range, 3-30; median 6.8 months in Phase 1, 12.7 months in Phase 2). The median follow-up since switching: 12.6 months (range, 1-25). Efficacy results of the MajesTEC-1 Q2W Cohort included the following:
At the time of switch, 54 (85.7%) patients achieved ≥CR (complete response or stringent complete response), 8 (12.7%) had VGPR, and 1 (1.6%) had PR.
68.7% (95% CI, 53.6-79.7) of patients who switched remained in response for ≥2 years from the time of first response (mDOR not yet reached)
Patients who switched (N=63), as of data cutoff of Jan. 4, 2023:
   42 maintained a response, 41 still on treatment;
   14 progressed after responding;
   5 died (4 due to COVID-19);
   1 had subsequent therapy after responding; and
   1 withdrew consent.

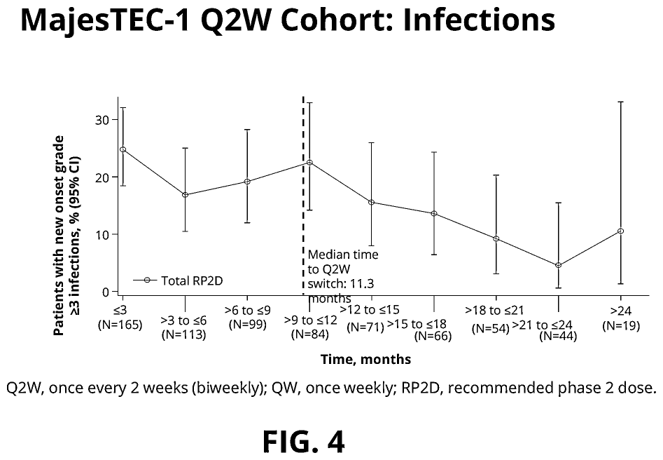

As shown in FIG. 4, There was a reduction in new onset grade ≥3 infections over time. Patients who switched to Q2W dosing by 1 year had fewer grade ≥3 treatment-emergent infections between 1-1.5 years than those who remained on QW dosing by 1 year (15.6% vs 33.3%).

In summary, at a data cut-off of Jan. 4, 2023, in the MajesTEC-1 Q2W cohort, most patients who switched to Q2W or Q4W teclistamab dosing maintained deep responses. Of the 104 responders, 63 switched from QW to Q2W or Q4W dosing, and 65% ( 41/63) remain on treatment; 68.7% of patients maintained their response for ≥2 years. There were fewer new onset grade ≥3 infections seen in patients who switched to Q2W or Q4W compared with those on QW teclistamab dosing.

It is noted that MajesTEC-1 has the longest follow-up (23 months) for a bispecific antibody in multiple myeloma, as of Jan. 4, 2023.

### Example 2: Evaluation of Prophylactic Tocilizumab in for the Reduction of Cytokine Release Syndrome

### Antibodies

Anti-BCMA/anti-CD3 antibody teclistamab (also called Tec) (e.g., described in WO2017031104A1, the content of which is incorporated herein by reference in its entirety) was made by Janssen Pharmaceuticals. Teclistamab comprises a BCMA binding arm BCMB69 and a CD3 binding arm CD3B219, the amino acid sequences of which are shown in Table 5 and Table 6 above, respectively.

Methods: Eligible pts were aged ≥18 y with RRMM and had previously received a PI, IMiD, and anti-CD38 antibody. All pts provided informed consent. Pts received subcutaneous teclistamab (following 2 step-up doses) in a prospective exploratory cohort at the RP2D or in a fixed-dose cohort. Toci (single 8 mg/kg IV dose) was given ≤4 hours before the first teclistamab step-up dose. CRS was graded per Lee et al 2014 and managed per institutional guidelines.

Initial Results: 14 pts were included: median age 64 y (range 50-82); all pts had ECOG score ≤1 and ISS I/II; 91% had standard cytogenetic risk; 21% had extramedullary plasmacytomas; 31% had ≥30% BMPCs; median 4 prior lines of therapy (range 2-7); 64% triple-class refractory. Median follow-up was 1.2 mo (range 0.2-4.6). CRS occurred in 4 pts (29%; no gr ≥3 CRS); 1 had a subsequent CRS event (table). Median time to onset was 2 d (range 1-3); median duration was 2 d (range 2-4); all events were managed with toci and resolved without teclistamab discontinuation. 7 of 14 pts had gr 3/4 neutropenia (consistent with the overall MajesTEC-1 population). 2/14 had a gr 3/4 infection. 2/14 had a neurotoxicity AE. Of the response evaluable patients, 4/7 responded. Cytokine and PK data in additional pts with longer follow-up will be presented to further inform pt management. Results are shown in the Table below.

TABLE
Grade, timing, and disease characteristics in pts experiencing
CRS with teclistamab following prophylactic toci.

| | CRS | Dose after which | Disease characteristics in pts with |
|---|---|---|---|
| Pt | grade | CRS occurred | CRS |
| 1 | 1 | Step-up dose 1 | 40% BMPCs (biopsy); 0 plasmacytomas |
| 2 | 2 | Step-up dose 2 | 30% BMPCs (aspirate); 2 plasmacytomas |
| 3 | 1 | Step-up dose 2 | 70% BMPCs (biopsy); 0 plasmacytomas; |
| | 2 | Cycle 1 Day 1 | 11.6% circulating plasma cells at |
| | | | baseline |
| 4 | 2 | Step-up dose 1 | 80% BMPCs (biopsy); 0 plasmacytomas |

BMPC, bone marrow plasma cell; CRS, cytokine release syndrome; pts, patients; toci, tocilizumab.

Follow-up Results: As of Apr. 28, 2023, 23 patients received prophylactic toci prior to teclistamab (median follow-up 2.6 months). Patients had received a median of 4 prior lines of therapy. Prophylactic toci reduced CRS incidence to 26.1% ( 6/23 patients):

two patients had Grade 1 CRS and 4 patients had Grade 2 CRS. No grade 3 or higher CRS was reported. Of the 16 response-evaluable patients, 2 (12.5%) had CR, 6 (37%) had VGPR, and 3 (18.8%) had PR. Prophylactic toci did not appear to affect the PK profile of teclistamab. Prophylactic toci reduced the overall incidence of CRS to 26% without impacting response to teclistamab, representing a 46% reduction in CRS observed in MajesTEC-1.

Summary/Conclusion: A single dose of toci before teclistamab treatment appeared to reduce the incidence of CRS relative to the overall MajesTEC-1 study population with no new safety signals and no evidence of impact on response to teclistamab considering the short follow-up. Prophylactic toci has the potential to reduce CRS risk in pts with disease profiles suitable for outpatient dosing, reducing the burden of hospitalization during teclistamab step-up dosing.

### Example 3: Model-Based Exploration of the Impact of Prophylactic Tocilizumab on IL-6 Dynamics in Multiple Myeloma Patients Receiving Teclistamab Treatment

A mechanism-based PK/PD model was used to evaluate the impact of tocilizumab prophylactic treatment on the soluble IL-6R (sIL-6R), IL-6 and the duration of the blockade of IL-6 signaling pathway in the patients following teclistamab treatment in the MajesTEC-1 cohort described in Example 2 above.

Methods: A mechanism-based PK/PD model was built by using reported tocilizumab PK parameters and sIL-6R target engagement. The time course of IL-6R RO for patients receiving prophylactic tocilizumab following teclistamab treatment was estimated from the PK profiles of tocilizumab and competitive binding kinetics. Among the patients with evaluable serum IL-6 levels, the highest peak serum IL-6 concentration in the patients who didn't have CRS (N=9) from prophylactic tocilizumab cohort of MajesTEC-1 was selected as the worst-case scenario. The IL-6R RO in patients receiving prophylactic tocilizumab in the worst-case scenario was characterized by model simulations and the results were compared to the calculated IL-6R RO in the cohorts following teclistamab treatment without prophylactic tocilizumab (N=40).

Results: Time course of serum IL-6 levels following teclistamab treatment showed higher and earlier induction in the cohort with tocilizumab prophylaxis compared to the cohort without tocilizumab prophylaxis. Among the patients who received tocilizumab prophylactic treatment, the median peak serum IL-6 level was 208.6 pg/mL and the highest peak serum IL-6 level was 1701.8 pg/mL in patients who didn't have CRS. The model simulation results showed that a single dose of tocilizumab at 8 mg/kg IV given prophylactically could lower the IL-6R RO by IL-6. In a patient with 1700 μg/mL of serum IL-6 treated with tocilizumab, IL-6R RO was maintained below the level of RO achieved with 10 μg/mL of serum IL-6 in the absence of tocilizumab for approximately 10 days. An IL-6 level of 10 μg/mL was considered as a representative value in the patients who did not have CRS following teclistamab at the approved dosing regimen without prophylactic tocilizumab, as more than half (58%) of these patients showed peak serum IL-6 level below 10 μg/mL. The duration of IL-6R RO reduction covers the two step-up doses (SUD) and the first full treatment dose according to the teclistamab approved dosing schedule.

Conclusions: The modeling and simulation results showed that a single dose of tocilizumab given prophylactically at 8 mg/kg could block IL-6R RO for approximately 10 days following the 1st step up dose of teclistamab. The results further support this approach for lowering overall risk of CRS during the SUD schedule in multiple myeloma patients treated with teclistamab.

### Example 4: A Phase 3 Randomized Study Comparing Teclistamab in Combination with Daratumumab SC (Tec-Dara) Versus Daratumumab SC, Pomalidomide, and Dexamethasone (DPd) or Daratumumab SC, Bortezomib, and Dexamethasone (DVd) in Participants with Relapsed or Refractory Multiple Myeloma (MajesTEC-3); ClinicalTrials.Gov ID NCT05083169)

### Antibodies

Daratumumab and teclistamab (also called Tec) (e.g., described in WO2017031104A1, the content of which is incorporated herein by reference in its entirety) are made by Janssen Pharmaceuticals. Teclistamab comprises a BCMA binding arm BCMB69 and a CD3 binding arm CD3B219, the amino acid sequences of which are shown in Table 5 and Table 6 above, respectively.

### Study Overview

The purpose of this study is to compare the efficacy of teclistamab daratumumab (Tec-Dara) with daratumumab subcutaneously (SC) in combination with pomalidomide and dexamethasone (DPd) or daratumumab SC in combination with bortezomib and dexamethasone (DVd).

The primary hypothesis of this study is that Tec-Dara will significantly improve progression free survival (PFS) compared with investigator's choice of DPd/DVd in participants with relapsed refractory multiple myeloma. Approximately 560 participants will be randomly assigned in a 1:1 ratio to receive either Tec-Dara (Arm A) or investigator's choice of DPd/DVd (Arm B). The study will be conducted in 3 phases: Screening Phase, Treatment Phase, and Follow-up Phase. Participants will be treated until disease progression, unacceptable toxicity, or other reasons to discontinue the study. Disease evaluation will occur every cycle. Safety will be assessed throughout the study. Efficacy will be assessed using International Myeloma Working Group (IMWG) criteria. The overall duration of the study will be approximately 5 years.

### Eligibility Criteria

Inclusion Criteria for this study include the following:
Documented multiple myeloma as defined by the criteria: a. multiple myeloma diagnosis according to the International Myeloma Working Group (IMWG) diagnostic criteria, b. measurable disease at screening as defined by any of the following: 1) serum M-protein level greater than or equal to (>=) 0.5 gram per deciliter (g/dL); or 2) urine M-protein level >=200 milligrams (mg)/24 hours; or 3) serum immunoglobulin free light chain >=10 mg/dL and abnormal serum immunoglobulin kappa lambda free light chain ratio
Received 1 to 3 prior line(s) of antimyeloma therapy including a proteasome inhibitor (PI) and lenalidomide; a. participants who have received only 1 line of prior line of antimyeloma therapy must be lenalidomide refractory. Stable disease or progression on or within 60 days of the last dose of lenalidomide given as maintenance will meet this criterion
Documented evidence of progressive disease based on investigator's determination of response by IMWG criteria on or after their last regimen
Have an eastern cooperative oncology group (ECOG) performance status score of 0, 1, or 2 at screening and prior to the start of administration of study treatment
Have clinical laboratory values within the specified range

Exclusion Criteria for this study include the following:
Contraindications or life-threatening allergies, hypersensitivity, or intolerance to any study drug or its excipients. Additional exclusion criteria pertaining to specific study drugs include:
   1. A participant is not eligible to receive daratumumab subcutaneous (SC) in combination with pomalidomide and dexamethasone (DPd) as control therapy if any of the following are present: 1) Contraindications or life-threatening allergies, hypersensitivity, or intolerance to pomalidomide, 2) Disease that is considered refractory to pomalidomide per IMWG,
   2. A participant is not eligible to receive daratumumab SC in combination with bortezomib and dexamethasone (DVd) as control therapy if any of the following are present: 1) Contraindications or life-threatening allergies, hypersensitivity, or intolerance to bortezomib, 2) Grade 1 peripheral neuropathy with pain or Grade >=2 peripheral neuropathy as defined by National Cancer Institute Common Terminology Criteria for Adverse Events (NCI-CTCAE) Version 5.0, 3) Disease that is considered refractory to bortezomib per IMWG, 4) Received a strong cytochromes P450 (CYP3A4) inducer within 5 half-lives prior to randomization
Received any prior B cell maturation antigen (BCMA)-directed therapy
Has disease that is considered refractory to an anti-cluster of differentiation 38 (CD38) monoclonal antibody per IMWG
Received a cumulative dose of corticosteroids equivalent to >=140 mg of prednisone within 14 days before randomization
Received a live, attenuated vaccine within 4 weeks before randomization
Plasma cell leukemia at the time of screening, Waldenström's macroglobulinemia, POEMS syndrome (polyneuropathy, organomegaly, endocrinopathy, M-protein, and skin changes), or primary amyloid light chain amyloidosis

### Arms and Interventions

Experimental Arm A: Teclistamab-daratumumab (Tec-Dara). Participants will receive teclistamab and daratumumab by subcutaneous (SC) injection. Step-up doses of teclistamab will be given prior to the first full dose.

Experimental Arm B: DPd or DVd. Participants will be randomized either to daratumumab, pomalidomide, dexamethasone (DPd) treatment to receive daratumumab SC injection; pomalidomide orally; dexamethasone orally or intravenously, or to Daratumumab, Bortezomib, Dexamethasone (DVd) treatment to receive daratumumab SC injection; bortezomib SC injection, and dexamethasone orally or intravenously.

### Treatment Dose and Route of Administration for Teclistamab

Teclistamab SC is administered in 28-day treatment cycles, as follows:
Cycle 1: 0.06 mg/kg step-up dose on Day 2, 0.3 mg/kg step-up dose on Day 4, 1.5 mg/kg step-up dose (also referred to as a treatment dose) on Day 8 and 1.5 mg/kg weekly (QW) thereafter (e.g., Days 15 and 22);
Cycle 2: 1.5 mg/kg weekly (QW) (e.g., Days 1, 8, 15 and 22);
Cycles 3-6: 3 mg/kg biweekly (Q2W) (e.g., Days 1 and 15);
Cycle 7 and subsequent cycles: 3 mg/kg monthly (Q4W) (e.g., Day 1).

Step-up dose 1 must be administered ≥20 hours after daratumumab SC given on C1D1. Thereafter, there must be ≥2 days between step-up doses of Tec. The 1.5 mg/kg treatment dose of teclistamab must be administered ≥2 days after Step-up Dose 2. Thereafter, there must be ≥5 days between 1.5 mg/kg treatment doses (or between a 1.5 mg/kg and the switch to the 3 mg/kg dose) and ≥12 days between 3 mg/kg treatment doses. On C1D8 (or the first time daratumumab and teclistamab are administered on the same day, in the case of dose delays), teclistamab should be administered ≥3 hours after daratumumab SC. If participant experiences treatment delays of daratumumab SC longer than 3 months, the interval between daratumumab SC and teclistamab should be at least 3 hours for the first resumed daratumumab dose.

### Justification for Dose:

As noted above, the teclistamab SC dose schedule is initiated with the RP2D dose, comprising 2 step-up doses (0.06 and 0.3 mg/kg) followed by a weekly treatment dose of 1.5 mg/kg. The step-up and 1.5 mg/kg treatment doses of teclistamab for Arm A were selected after review of safety, efficacy, PK, and pharmacodynamic data from participants with relapsed or refractory multiple myeloma treated with this regimen as a monotherapy in Study 64007957MMY1001. No DLTs were observed during the dose escalation phase for this dose level. CRS was generally low grade, and the median duration of CRS was 2 days. Neurotoxicity events were uncommon and all low grade. Cytopenias were common and manageable. Among the 150 participants treated at 1.5 mg/kg weekly, ORR was robust, with responses occurring rapidly and deepening over time.

Trough levels following the 1.5 mg/kg SC injections are comparable to the max EC90 of the ex-vivo cytotoxicity assay. This assay assessed the ability of teclistamab to induce killing using mononuclear cells from the bone marrow samples of multiple myeloma patients in co-culture with T cells from healthy donors. Pharmacodynamic data also support this regimen with induction of cytokines and T cell activation following step-up doses indicative of the mechanism of action.

In this study, participants will receive teclistamab SC Q2W starting on Cycle 3 Day 1 at a treatment dose of 3 mg/kg (implemented with a protocol amendment). This change in dose schedule from 1.5 mg/kg weekly to 3 mg/kg Q2W will increase convenience and flexibility for patients, caregivers, and healthcare providers. Given the PK profile of teclistamab described below, it is estimated that the 3 mg/kg Q2W dose schedule will provide comparable exposure to that of 1.5 mg/kg weekly, with adequate exposure over the intended dosing interval.

In MajesTEC-1, teclistamab exposure increased in a dose-proportional manner over the range of 0.08 to 6 mg/kg weekly SC administration. Given the robust PK data and the tolerable safety data in all cohorts evaluated to date, this SC dose schedule of 1.5 mg/kg teclistamab weekly for 2 cycles and 3 mg/kg teclistamab Q2W in Cycles 3-6 is estimated to provide comparable exposure with the 1.5 mg/kg teclistamab weekly dose schedule. Consequently, this dose schedule is calculated to achieve comparable efficacy and tolerable safety.

The population PK analysis used serum teclistamab concentration data from MajesTEC-1 with the PK data cutoff of 14 Jun. 2021. The final dataset included a total of 4143 measurable serum teclistamab concentrations from 308 PK-evaluable adult participants (n=83 received IV dose; n=225 received SC dose) with relapsed or refractory multiple myeloma. The weight distribution in this study population ranged from 41 to 139 kg, with a median body weight of 74 kg. The final population PK model was used to simulate teclistamab concentration-time profiles. The participants for simulation were randomly sampled from the analysis dataset (n=1000).

The model estimates that 3 mg/kg teclistamab SC Q2W dosing will result in majority of participants with C_{trough} above the maximum EC90 (target exposure), which was comparable with 1.5 mg/kg teclistamab SC weekly dosing. Following 3 mg/kg teclistamab SC Q2W dosing on Week 20, the model estimates that C_{trough}, Cₘₐₓ, and AUC_{0-14 days} to be comparable with those of 1.5 mg/kg teclistamab SC weekly dosing. It is estimated that the accumulation ratio following teclistamab SC Q2W dosing will be less than half of weekly 1.5 mg/kg teclistamab SC dosing.

Following 3 mg/kg teclistamab SC Q4W dosing, the model estimated steady-state median C_{trough,ss}, C_{max,ss}, and AUC_{ss, 0-28 day} are lower than those of 1.5 mg/kg teclistamab SC weekly dosing. However, the median trough levels following 3 mg/kg Q4W are estimated to be comparable to the max EC90. In addition, 37 participants in MajesTEC-1 switched to less frequent dosing, with all responding prior to the switch. As of 16 Mar. 2022, these participants had a median duration of maintaining response after the dosing frequency change of 12.9 months (range 1 to 18 months), with 28 participants (76%) remaining in response. It should be noted that in Majes-TEC-1, a majority of the responders (approximately 90%) are at VGPR or better (median time to VGPR or better: 2.1 months, range 0.2-10.3 months) by Cycle 7; thus, this predicted exposure should be sufficient to maintain efficacy.

Notably, the safety profile of teclistamab in combination with daratumumab SC in Study 64407564MMY1002 (including teclistamab 3 mg/kg Q2W dose schedule) appears to be consistent with those of the individual study drugs with no deleterious effects on the incidence or severity of CRS, sARR, neurotoxicity or other potential clinically important overlapping toxicities (i.e., injection-site reactions, cytopenias, or infections). Response data in this study are preliminary but suggest a promising effect of teclistamab in combination with daratumumab SC. Overall, it is estimated that teclistamab SC dose schedule of 1.5 mg/kg weekly for 2 cycles and 3 mg/kg Q2W followed by 3 mg/kg teclistamab Q4W in Cycle 7 will have comparable safety and efficacy with the 1.5 mg/kg teclistamab weekly dose schedule, and further increase the convenience and flexibility for patients, caregivers, and healthcare providers.

### Treatment Dose and Route of Administration for Other Study Drugs

The dose schedule for daratumumab SC is outlined below; pomalidomide or bortezomib; and dexamethasone are based on the standard doses used in clinical practice for the treatment of multiple myeloma, as detailed in the respective local prescribing information.

Daratumumab SC (1800 mg) is administered in 28-day treatment cycles, as follows:
Cycle 1 Day 1 and weekly (QW) thereafter (e.g., Days 1, 8, 15 and 22);
Cycle 2: weekly (QW) (e.g., Days 1, 8, 15 and 22);
Cycles 3-6: biweekly (Q2W) (e.g., Days 1 and 15);
Cycle 7 and subsequent cycles: monthly (Q4W) (e.g., Day 1).

There must be ≥5 days between doses of daratumumab SC.

### Outcome Measures

The primary endpoint is progression-free survival (PFS), defined as the time from the date of randomization to the date of first documented disease progression, as defined in the IMWG criteria, or death due to any cause, whichever occurs first. Response to treatment will be assessed by the IRC, investigator, and validated computerized algorithm; assessment by the IRC will be used as primary.

The secondary endpoints will be summarized as:

Overall response (PR or better) is defined as participants who have a PR or better per IMWG criteria.

VGPR or better (sCR+CR+VGPR) is defined as participants who achieve a VGPR or better response per IMWG criteria.

CR or better (sCR+CR) is defined as participants who achieve a CR or better response per IMWG criteria.

MRD negativity is defined as participants who achieve MRD negativity at a threshold of 10⁻⁵ at any timepoint after the date of randomization and before disease progression or start of subsequent antimyeloma therapy.

Progression Free Survival on Next-line Therapy (PFS2) is defined as the time interval between the date of randomization and date of event, which is defined as antimyeloma therapy, or death from any cause, whichever occurs first. Those who are alive and for whom a second disease progression has not been observed are censored at the last date of follow-up.

Overall Survival (OS) is measured from the date of randomization to the date of the participant's death. If the participant is alive or the vital status is unknown, then the participant's data will be censored at the date the participant was last known to be alive.

Time to Next Treatment (TTNT) is defined as the time from randomization to the start of subsequent antimyeloma treatment. Death due to PD without start of subsequent therapy will be considered as event. Participants who withdrew consent to study, are lost to follow-up, or die due to causes other than disease progression will be censored at the date of death or the last date known to be alive.

Duration of Response will be calculated among responders (with a PR or better response) from the date of initial documentation of a response (PR or better) to the date of first documented evidence of progressive disease according to the IMWG response criteria, or death due to any cause, whichever occurs first. Participants who have not progressed and are alive will be censored at the last disease evaluation before the start of subsequent anti-myeloma therapy.

What is claimed is:

1-100. (canceled)

101. A method of treating multiple myeloma in a subject in need thereof, comprising subcutaneously administering to the subject treatment doses of a BCMAxCD3 bispecific antibody on a weekly dosing schedule (QW) for at least one 28-day treatment cycle, and subsequently administering to the subject treatment doses of the BCMAxCD3 bispecific antibody on a bi-weekly dosing schedule (Q2W) for at least one 28-day treatment cycle, and subsequently administering to the subject treatment doses of the BCMAxCD3 bispecific antibody on a monthly dosing schedule (Q4W),
wherein the BCMAxCD3 bispecific antibody comprises a BCMA binding domain comprising the HCDR1 of SEQ ID NO: 4, the HCDR2 of SEQ ID NO: 5, the HCDR3 of SEQ ID NO: 6, the LCDR1 of SEQ ID NO: 7, the LCDR2 of SEQ ID NO: 8 and the LCDR3 of SEQ ID NO: 9, and a CD3 binding domain comprising the HCDR1 of SEQ ID NO: 14, the HCDR2 of SEQ ID NO: 15, the HCDR3 of SEQ ID NO: 16, the LCDR1 of SEQ ID NO: 17, the LCDR2 of SEQ ID NO: 18 and the LCDR3 of SEQ ID NO: 19, and
wherein the method is effective in treating the multiple myeloma.

102. The method of claim 101, wherein the BCMA binding domain comprises a heavy chain variable region (VH) having the amino acid sequence of SEQ ID NO: 10 and a light chain variable region (VL) having the amino acid sequence of SEQ ID NO: 11, and the CD3 binding domain comprises a heavy chain variable region (VH) having the amino acid sequence of SEQ ID NO: 20 and a light chain variable region (VL) having the amino acid sequence of SEQ ID NO: 21.

103. The method of claim 101, wherein the BCMAxCD3 bispecific antibody comprises a first heavy chain (HC1) having the amino acid sequence of SEQ ID NO: 12, a first light chain (LC1) having the amino acid sequence of SEQ ID NO: 13, a second heavy chain (HC2) having the amino acid sequence of SEQ ID NO: 22 and a second light chain (LC2) having the amino acid sequence of SEQ ID NO: 23.

104. The method of claim 101, wherein the BCMAxCD3 bispecific antibody is teclistamab.

105. The method of claim 101, wherein the subject has relapsed or refractory multiple myeloma.

106. The method of claim 101, comprising subcutaneously administering to the subject one or more step-up doses of the BCMAxCD3 bispecific antibody prior to administering the first treatment dose of the BCMAxCD3 bispecific antibody.

107. The method of claim 101, comprising administering the BCMAxCD3 bispecific antibody on the weekly dosing schedule (QW) at a treatment dose of about 1500 μg/kg, and administering the BCMAxCD3 bispecific antibody on the bi-weekly dosing schedule (Q2W) at a treatment dose of about 3000 μg/kg.

108. The method of claim 101, comprising administering the BCMAxCD3 bispecific antibody on the weekly dosing schedule (QW) at a treatment dose of about 1500 μg/kg, administering the BCMAxCD3 bispecific antibody on the bi-weekly dosing schedule (Q2W) at a treatment dose of about 3000 μg/kg, and administering the BCMAxCD3 bispecific antibody on the monthly dosing schedule (Q4W) at a treatment dose of about 3000 μg/kg.

109. The method of claim 101, wherein the subject achieves a clinical response that is a partial response (PR), or a very good partial response (VGPR), or a complete response (CR) or a stringent complete response (sCR) according to International Myeloma Working Group (IMWG) criteria.

110. The method of claim 101, comprising administering a combination regimen comprising the BCMAxCD3 bispecific antibody and daratumumab.

111. The method of claim 101, comprising administering the BCMAxCD3 bispecific antibody in the following 28-day treatment cycles:
Cycle 1: 0.06 mg/kg step-up dose on Day 2, 0.3 mg/kg step-up dose on Day 4, 1.5 mg/kg step-up dose (also referred to as a treatment dose) on Day 8 and 1.5 mg/kg weekly (QW) thereafter;
Cycle 2: 1.5 mg/kg weekly (QW);
Cycles 3-6: 3 mg/kg biweekly (Q2W);
Cycle 7 and subsequent cycles: 3 mg/kg monthly (Q4W).

112. The method of claim 104, wherein the subject has relapsed or refractory multiple myeloma.

113. The method of claim 104, comprising subcutaneously administering to the subject one or more step-up doses of the BCMAxCD3 bispecific antibody prior to administering the first treatment dose of the BCMAxCD3 bispecific antibody.

114. The method of claim 104, comprising administering the BCMAxCD3 bispecific antibody on the weekly dosing schedule (QW) at a treatment dose of about 1500 μg/kg, and administering the BCMAxCD3 bispecific antibody on the bi-weekly dosing schedule (Q2W) at a treatment dose of about 3000 μg/kg.

115. The method of claim 104, comprising administering the BCMAxCD3 bispecific antibody on the weekly dosing schedule (QW) at a treatment dose of about 1500 μg/kg, administering the BCMAxCD3 bispecific antibody on the bi-weekly dosing schedule (Q2W) at a treatment dose of about 3000 μg/kg, and administering the BCMAxCD3 bispecific antibody on the monthly dosing schedule (Q4W) at a treatment dose of about 3000 μg/kg.

116. The method of claim 104, wherein the subject achieves a clinical response that is a partial response (PR), or a very good partial response (VGPR), or a complete response (CR) or a stringent complete response (sCR) according to International Myeloma Working Group (IMWG) criteria.

117. The method of claim 104, comprising administering a combination regimen comprising the BCMAxCD3 bispecific antibody and daratumumab.

118. The method of claim 104, comprising administering the BCMAxCD3 bispecific antibody in the following 28-day treatment cycles:
Cycle 1: 0.06 mg/kg step-up dose on Day 2, 0.3 mg/kg step-up dose on Day 4, 1.5 mg/kg step-up dose (also referred to as a treatment dose) on Day 8 and 1.5 mg/kg weekly (QW) thereafter;
Cycle 2: 1.5 mg/kg weekly (QW);
Cycles 3-6: 3 mg/kg biweekly (Q2W);
Cycle 7 and subsequent cycles: 3 mg/kg monthly (Q4W).
